(19) United States
(12) Patent Application Publication
(10) Pub. No.: US 2011/0212101 A1
(43) Pub. Date: Sep. 1, 2011

(54) MATERIALS AND METHODS FOR EXPLOITING SYNTHETIC LETHALITY IN MISMATCH REPAIR-DEFICIENT CANCERS

(76) Inventors: Sarah Martin, Greater London (GB); Christopher James Lord, Greater London (GB); Alan Ashworth, Greater London (GB)

(21) Appl. No.: 12/674,734

(22) PCT Filed: Aug. 22, 2008
(86) PCT No.: PCT/GB2008/002850

§ 371 Date: May 10, 2011

Related U.S. Application Data
(60) Provisional application No. 60/957,813, filed on Aug. 24, 2007.

(51) Int. Cl. A61K 39/395; C12Q 1/18; C12Q 1/48; C12Q 1/26; A61P 35/00

(52) U.S. Cl. 424/158.1; 435/32; 435/15; 435/25

(57) ABSTRACT

Therapeutic approaches to the treatment of DNA mismatch repair (MMR) deficient cancers are disclosed based on the use of complimentary gene-function and drug screening synthetic lethality approaches for designing therapies for the treatment of cancers where loss of tumour suppressor function has occurred. The work is based on experiments using human MSH2, an integral component of the MMR pathway, and is applicable to other genes in the MMR pathway, and in particular MLH1, MSH6, PMS1 and PMS2. In particular loss of MSH2 is synthetically lethal with inhibition of the DNA polymerase POLβ deficiency of MLH1 is synthetically lethal with DNA polymerase γ (POLG) inhibition.

### FIELD OF THE INVENTION

The present invention relates to materials and methods for exploiting synthetic lethality in DNA mismatch repair (MMR) deficient cancers, including the treatment of cancer and screening candidate compounds for use in treating cancer.

### BACKGROUND OF THE INVENTION

Each year, the majority of new cancer drug approvals are directed against existing targets, whereas only two or three compounds are licensed against novel molecules. Rather than suggesting a limiting number of targets, this reflects the difficulty, time and cost involved in the identification and validation of proteins that are crucial to disease pathogenesis. The result is that many key proteins remain undrugged, and as a consequence opportunities to develop novel therapies are lost. This situation could be improved by using approaches that identify the key molecular targets that underlie the pathways that are associated with disease development. For example, techniques such as gene targeting, in which a gene can be selectively inactivated or knocked-out, can be powerful. However, such approaches are limited by their cost and low throughput.

Moreover, it is often the case that the current approaches to cancer treatment group together similar clinical phenotypes regardless of the differing molecular pathologies that underlie them. A consequence of this molecular heterogeneity is that individuals frequently exhibit vast differences to drug treatments. As such, therapies that target the underlying molecular biology of individual cancers are increasingly becoming an attractive approach.

The DNA mismatch repair (MMR) pathway is integral in the maintenance of genomic stability. MMR functions in postreplicative repair by correcting DNA polymerase errors including base-base or insertion/deletion mismatches that form during DNA replication. Mutations in MMR genes are often associated with an increase in the frequency of spontaneous mutation and carcinogenesis (Jascur and Boland, 2006 and Jiricny, 2006). Defects in MMR are often characterised by microsatellite instability (MSI) caused by expansion or contraction of short nucleotide repeats in the absence of efficient MMR (Ionov et al, 1993; Aaltonen et al, 1993) and, as such, MSI is detectable in the majority of colorectal cancers arising in carriers of germ-line MMR mutations (Aaltonen et al., 1994; Liu et al., 1996).

Mutations in two of the MMR genes, MSH2 and MLH1, segregate with disease in ˜50% of families with hereditary non-polyposis colorectal cancer (HNPCC), which accounts for approximately 5% of all colorectal cancer cases (Jacob and Praz, 2002) (Liu B, Parsons R, Papadopoulos N, et al. Nat Med 1996; 2:169-74 and Kolodner R D, Tytell J D, Schmeits J L, et al. Cancer Res 1999; 59:5068-74 and Wijnen J T, Vasen H F, Khan P M, et al. N Engl J Med 1998; 339:511-8.). A minority of HNPCC families have colorectal cancer due to mutations in other MMR genes, such as MSH6 (Farrington S M, Lin-Goerke J, Ling J, et al. Am J Hum Genet 1998; 63:749-59). Inactivation of the remaining wild-type allele in MLH1 and MSH2 mutant tumours has been shown to occur by somatic muation (Cunningham et al., 2001, Leach et al., 1993), loss of heterozygosity (LOH; Yuen et al., 2002, Potocnik et al., 2001) or promoter hypermethylation (Cunningham et al., 1998, Potocnik et al., 2001) suggesting that MLH1 and MSH2 act as classical tumour suppressor genes. Furthermore, germ-line mutations in MSH2 or MLH1 have been documented in non-familial cases of colorectal cancer, especially in individuals diagnosed with colorectal cancer at a young age (Vasen H F, Watson P, Mecklin J P, Lynch H T. Gastroenterology 1999; 116:1453-6.). More importantly, deficiency of MMR also plays a role in the development of colorectal cancer outside the context of HNPCC. Approximately 12% of all colorectal cancers (Aaltonen L A, Peltomaki P, Mecklin J P, et al. Cancer Res 1994; 54:1645-8), especially those developing in the proximal colon (Thibodeau SN, Bren G, Schaid D. Science 1993; 260:816-9.), exhibit MSI and defects in MMR are also observed in 10-25% of sporadic cancers, often as a result of aberrant MLH1 promoter methylation (Arnold et al., 2003, Bettstetter et al., 2007, Peltomaki, 2003).

### SUMMARY OF THE INVENTION

Broadly, the present invention is based on novel therapeutic approaches to the treatment of DNA mismatch repair (MMR) deficient cancers based on the use of complimentary gene-function and drug screening synthetic lethality approaches for designing therapies for the treatment of cancer's where loss of tumour suppressor function has occurred. These results are based on exemplary experiments involving hereditary nonpolyposis colorectal cancer (HNPCC) which is inherited as a dominant disorder caused by germline defects in DNA mismatch repair (MMR), a process that normally repairs errors that occur during DNA replication. The work is based on experiments using human MSH2, an integral component of the MMR pathway, but it is believed that the results are applicable to other genes in the MMR pathway, and in particular MLH1, MSH6, PMS1 and PMS2. In the work leading to the present invention, a synthetic lethal approach was employed to identify MSH2-selective therapeutic targets with a view to the design of new strategies for the treatment of MMR-deficient cancers. This demonstrated that loss of MSH2 is synthetically lethal with inhibition of the DNA polymerase POLβ and that this lethality is characterised by an accumulation of 8-hydroxy-2-deoxyguanosine (8-OHdG) DNA lesions. Similarly, deficiency of MLH1 is synthetically lethal with DNA polymerase γ (POLG) inhibition. MSH2 deficiency leads to POLB upregulation, while MLH1 deficiency is associated with POLG upregulation, suggesting that deficiencies in particular MMR proteins can be compensated for by upregulation of specific DNA polymerases. A combination of MSH2/POLB deficiencies results in accumulation of nuclear 8-OHdG lesions, and a combination of MLH1/POLG deficiencies results in accumulation of 8-OHdG lesions in mitochondria. POLB deficiency likely contributes to the accumulation of 8-OHdG lesions by causing a reduction in OGG1 expression. Furthermore, methods for identifying compounds suitable for use in the treatment of MMR-deficient cancer are provided, that can, for example, be used in high-throughput screening of compound libraries. The work disclosed herein also shows that agents that induce 8-OHdG accumulation, such as methotrexate, are synthetically lethal with MSH2 deficiency. Given the MMR/colorectal cancer relationship and the frequency of MMR defects in other tumourigenic conditions, these synthetic lethal relationships suggest novel therapeutic approaches.

Accordingly, in a first aspect, the present invention provides the use of an inhibitor of DNA polymerase POLβ or DNA polymerase POLy for the preparation of a medicament for the treatment of an individual having a DNA mismatch repair (MMR) deficient cancer.

In a further aspect, the present invention provides the use of an inhibitor of DNA polymerase POLβ, DNA polymerase POLy, telomerase transcriptional element integrating factor (TEIF or SCYL1) and/or dihydrofolate reductase (DHFR) for the preparation of a medicament for the treatment of an individual having a DNA mismatch repair (MMR) deficient cancer.

In a further aspect, the present invention provides the use of an agent that induces formation of 8-hydroxy-2′-deoxyguanosine (8-OHdG) lesions in cancer cells for the preparation of a medicament for the treatment of an individual having a DNA mismatch repair (MMR) deficient cancer. The formation of 8-OHdG lesions in cancer cells may be determined using assays well known in the art, such as the ELISA assay the use of which is exemplified herein. Generally, the formation of lesions is associated with an increase in the level of 8-OHdG in the cancer cells, for example as compared to the basal level caused by the normal metabolism of the cell.

In a further aspect, the present invention provides the use of methotrexate, parthenolide or menadione, or derivatives thereof, for the preparation of a medicament for the treatment of an individual having a DNA mismatch repair (MMR) deficient cancer.

In a further aspect, the present invention provides an inhibitor of DNA polymerase POLβ or DNA polymerase POLy for treating an individual having a DNA mismatch repair (MMR) deficient cancer.

In a further aspect, the present invention provides an inhibitor of DNA polymerase POLβ, DNA polymerase POLy, telomerase transcriptional element integrating factor (TEIF or SCYL1) and/or dihydrofolate reductase (DHFR) for treating an individual having a DNA mismatch repair (MMR) deficient cancer.

In a further aspect, the present invention provides an agent that induces formation of 8-hydroxy-2′-deoxyguanosine (8-OHdG) lesions in cancer cells for treating an individual having a DNA mismatch repair (MMR) deficient cancer.

In a further aspect, the present invention provides a method of treating an individual having a DNA mismatch repair (MMR) deficient cancer, the method comprising administering a therapeutically effective amount of an inhibitor of DNA polymerase POLβ or DNA polymerase POLy to the individual.

In a further aspect, the present invention provides a method of treating an individual having a DNA mismatch repair (MMR) deficient cancer, the method comprising administering a therapeutically effective amount of an inhibitor of DNA polymerase POLβ, DNA polymerase POLy, telomerase transcriptional element integrating factor (TEIF or SCYL1) and/or dihydrofolate reductase (DHFR) to the individual.

In a further aspect, the present invention provides a method of treating an individual having a DNA mismatch repair (MMR) deficient cancer, the method comprising administering a therapeutically effective amount of an agent that induces formation of 8-hydroxy-2′-deoxyguanosine (8-OHdG) lesions in cancer cells to the individual.

In the medical uses and methods of treatment that form part of the present invention, the individual having a MMR-deficient cancer may have a mutation in a gene in the MMR pathway. Examples of such genes include the MSH2 gene, the MLH1 gene, MSH6 gene, the PMS1 gene or the PMS2 gene. The mutations may be spontaneous or inherited. The full names and database accession information for the preferred genes in the MMR pathway are as follows:

MSH2 (mutS homolog 2, colon cancer, nonpolyposis type 1) 4436;
MSH6 (mutS homolog 6) 2956;
MLH1 (mutL homolog 1, colon cancer, nonpolyposis type 2) 4292;
PMS1 (PMS1 postmeiotic segregation increased) 5378; and
PMS2 (PMS2 postmeiotic segregation increased 2) 5395.

The full names and database accession information for the preferred target genes that can be inhibited to cause a synthetic lethal effect in a MMR-deficient cancer are as follows:

POLB (polymerase (DNA directed), beta) 5423;
POLγ (polymerase(DNA directed), gamma) 5428;
SCYL1 (SCY1-like 1) 57410; and
DHFR (dihydrofolate reductase) dihydrofolate reductase 1719.

Alternatively or additionally, the MMR-deficient cancer may be characterised by defects or inactivation of the MMR pathway that are associated with the cancer cells as opposed to the patient's non-cancerous cells. By way of example, the MMR-deficient cancer may be characterised by the cancer cells having a defect in DNA mismatch repair, the cancer cells exhibiting epigenetic inactivation of MSH2 or loss of MSH2 function, for example promoter hypermethylation that may be determined by methylation specific PCR to detect silencing of MMR genes. Examples of MMR-deficient cancer include colorectal cancer, such as non-polyposis colorectal cancer (HNPCC) or sporadic colorectal cancer, endometrial tumours, stomach tumours or transitional cell carcinoma of the urinary tract, childhood onset haematological or brain malignancy or Muir-Torre Syndrome. Also, the presence of MSH2 mutations in patients with hepatocellular carcinoma has been shown to correlate with poor prognosis and may serve as an indicator for poor survival in patients (Yano et al Eur. J. Cancer. 2007 April 43(6):1092-100.)

In a further aspect, the present invention provides a method of screening for agents useful in the treatment of a DNA mismatch repair (MMR) pathway deficient cancer, the method employing first and second cell lines, wherein the first cell line is deficient in a component of the DNA mismatch repair (MMR) pathway and the second cell line is proficient for said component of the DNA mismatch repair (MMR) pathway, the method comprising:
(a) contacting the first and second'cell lines with at least one candidate agent;
(b) determining the amount of cell death in the first and second cell lines; and
(c) selecting a candidate agent which is synthetically lethal in the first cell line.

In this method, it is preferable that the first and second cells lines are isogenically matched. It is also preferred that the cell lines are cancer cell lines, for example a human endometrial adenocarcinoma cell line, such as Hec59 used in the examples. The use of human cell lines or those from animal models (e.g. murine or rat) are preferred.

Alternatively or additionally, in a further aspect, the present invention provides a method of screening for agents useful in the treatment of a DNA mismatch repair (MMR) pathway deficient cancer, the method comprising:
(a) contacting a protein target with at least one candidate agent, wherein the protein target is selected from DNA polymerase POLβ, DNA polymerase POLy, telomerase transcriptional element integrating factor (TEIF or SCYL1) and/or dihydrofolate reductase (DHFR);
(b) determining an effect of at least one candidate agent on an activity of the protein target; and
(c) selecting a candidate agent that inhibits the activity of the protein target.

Alternatively or additionally, in a further aspect, the present invention provides a method of screening for agents useful in the treatment of a DNA mismatch repair (MMR) pathway deficient cancer, the method comprising:
(a) contacting a cell line deficient in a component of the DNA mismatch repair (MMR) pathway with at least one candidate agent;
(b) determining whether the candidate agent causes accumulation of 8-OHdG in the cell-line; and
(c) selecting a candidate compound that causes accumulation of 8-OHdG in the cell line.

As set out in detail below, candidate agents identified using a method of screening according to the present invention may be the subject of further development to optimise their properties, to determine whether they work well in combination with other chemotherapy or radiotherapy, to manufacture the agent in bulks and/or to formulate the agent as a pharmaceutical composition.

Embodiments of the present invention will now be described in more detail by way of example and not limitation with reference to the accompanying figures.

### BRIEF DESCRIPTION OF THE FIGURES

FIG. 1. hMSH2 is synthetically lethal with POLβ.

A. Western blots of lysates from Hec59+Chr2 and Hec59 cells 48 hours after transfection with siRNA oligonucleotides as indicated.
B. Hec59+Chr2 and Hec59 cells were transfected with siRNA oligonucleotides directed against POLβ as indicated in the graph. After 6 days, cells were analysed for cellular survival using an ATP assay by staining with CellTitre Glo. *-p≦0.0069 compared to the similarly transfected MSH2 proficient Hec59+chr2 cells (Student's t-test). Error bars represent standard errors of the mean.
C. Clonogenic survival of Hec59+Chr2 and Hec59 cells transfected with siRNA oligonucleotides as indicated in the graph. *-p≦0.0144 compared to the similarly transfected MSH2 proficient Hec59+chr2 cells (Student's t-test). Error bars represent standard errors of the mean.

FIG. 2. MSH2 deficiency is associated with increased POLβ expression.

A. Western blots of lysates from Hec59 and Hec59+chr2 cells were immunoblotted with indicated antibodies.
B. Increase of POL beta mRNA levels in the absence of MSH2 expression. mRNA levels were analysed by qRT-PCR with beta-actin used as a control. Error bars represent standard errors of the mean.
C. Western blot analysis of Hec59 and Hec59+chr2 cells transfected with either siControl siRNA or SCYL1 siRNA, as indicated. Protein lysates were immunoblotted with indicated antibodies.
D. Hec59+Chr2 and Hec59 cells were transfected with siRNA oligonucleotides directed against SCYL1 as indicated in the graph. After 6 days, cells were analysed for cellular survival using an ATP assay by staining with CellTitre Glo. *-p≦≦0.001 compared to the similarly transfected MSH2 proficient Hec59+chr2 cells (Student's t-test). Error bars represent standard errors of the mean.

FIG. 3. Increased 8-OHdG accumulation correlates with POLβ and MSH2 deficiency

Hec59 and Hec59+chr2 cells were transfected with siControl and POLβ siRNA. Isolated DNA from transfected cells were analysed for 8-OHdG accumulation using a specific 8-OHdG ELISA assay. Oxidised lesions were quantified according to an 8-OHdG standard curve. Assays were performed in triplicate. *-p≦0.0101 compared to the similarly transfected MSH2 proficient Hec59+chr2 cells (Student's t-test). Error bars represent standard errors of the mean.

FIG. 4. Small molecules inducing oxidative damage cause synthetic lethality with MSH2 deficiency

A. Hec59 and Hec59+chr2 cells were screened by high-throughput, using a library of 1120 compounds. The graph represents the log 2 ratio of cellular viability normalised to the equimolar DMSO treated samples.
B. Survival curves of Hec59 and Hec59+Chr2 cells under continuous exposure to a range of concentrations of Parthenolide, Menadione and Methotrexate for 14 days. Error bars represent standard errors of the mean.
C. Hec59 and Hec59+chr2 cells were treated with compounds as indicated. Isolated DNA from treated cells were analysed for 8-OHdG accumulation using a specific 8-OHdG ELISA assay. Oxidised lesions were quantified according to an 8-OHdG standard curve. Assays were performed in triplicate. *-p≦0.0327 compared to the similarly transfected MSH2 proficient Hec59+chr2 cells (Student's t-test). Error bars represent standard errors of the mean. Survival curves represent Hec59+chr2 cells (D) and HeLa cells (E) transfected with either siControl or MSH2 siRNA, under continuous exposure to a range of concentrations of Methotrexate for 14 days. Error bars represent standard errors of the mean.

FIG. 5. Methotrexate treatment is synthetically lethal with MSH2 deficiency through inhibition of folate synthesis.

A. Hec59+Chr2 and Hec59 cells were transfected with siRNA oligonucleotides directed against DHFR as indicated in the graph. After 6 days, cells were analysed for cellular survival using an ATP assay by staining with CellTitre Glo. *-p≦50.001 compared to the similarly transfected MSH2 proficient Hec59+chr2 cells (Student's t-test). Error bars represent standard errors of the mean.
B. Western blot analysis of Hec59 and Hec59+chr2 cells transfected with either siControl siRNA or DHFR siRNA, as indicated. Protein lysates were immunoblotted with indicated antibodies.
C. Survival curves represent Hec59 and Hec59+chr2 cells under continuous exposure to a range of concentrations of Methotrexate for 14 days, in the presence or absence of Folic Acid. Error bars represent standard errors of the mean.
D. Western blots of lysates from Hec59+Chr2 and Hec59 cells, 72 hours after treatment with methotrexate or transfected with POLβ siRNA were immunoblotted as indicated.

FIG. 6. Reduction in POLβ expression upon methotrexate treatment

A. Western blots of lysates from HeC59+Chr2 and Hec59 cells, 72 hours after treatment with compounds as indicated.
B. Expression data taken from GEO omnibus accession no. GDS330 representing paired primary acute lymphoblastic leukemia patients samples comparing pre-treatment controls with samples treated with high-dose methotrexate. Statistical significance of expression was determined by Students t-test (*-p≦0.0001).
C. POLβ expression data from pre-treatment controls, samples treated with high-dose methotrexate and samples treated with mercaptopurine were downloaded from GEO omnibus accession no. GDS330 and analysed. Statistical significance of expression was determined by ANOVA (*-p<0.0001).

FIG. 7. MMR deficiencies are synthetically lethal with silencing of DNA polymerases

A. Deficiency in MSH2 is synthetically lethal with POLB inhibition. Hec59 (MSH2 deficient) and Hec59+Chr2 (MSH2 proficient) cells were transfected with siRNA oligonucleotides directed against various DNA Polymerases as indicated in the graph. After five days, cell viability was assessed. Error bars represent standard errors of the mean.
B. Cell lysates from Hec59 and Hec59+Chr2 cells were analysed by western blotting. Antibodies directed against MSH2 and β-tubulin were used to demonstrate presence or absence of MSH2 expression in both cell lines.
C. Deficiency in MLH1 is synthetically lethal with POLG inhibition. HCT116 (MLH1 deficient) and HCT116+Chr3 (MLH1 proficient) cells were transfected with siRNA oligonucleotides directed against various DNA Polymerases as indicated in the graph. After 5 days, cells were analysed for cellular survival using an ATP assay by staining with CellTitre Glo. Error bars represent standard errors of the mean.
D. Cell lysates from HCT116 and HCT116+CHR3 cells were analysed by western blotting. Antibodies directed against MLH1 and 13-tubulin, were used to demonstrate presence or absence of MLH1 expression in both cell lines.

FIG. 8. MMR deficiency is associated with increased DNA polymerase expression

A. Elevated POLB mRNA levels in the absence of MSH2 expression. POLB mRNA levels were analysed by qRT-PCR with β-actin used as a control. *-p=0.0254 compared to the MSH2 proficient Hec59+chr2 cells (Student's t-test). Error bars represent standard errors of the mean.

FIG. 9. Increased 8-OHdG accumulation correlates with selective lethality with MSH2 deficiency

A. Increased 8-OHdG accumulation upon MSH2 deficiency and silencing of POLB and OGG1. Hec59 and Hec59+chr2 cells were transfected with Control, POLB and OGG1 siRNA. Isolated DNA from transfected cells were analysed for 8-OHdG accumulation using a 8-OHdG ELISA assay. Oxidised lesions were quantified according to a standard curve generated using known amounts of 8-OHdG. Assays were performed in triplicate. *-p≦0.0101 compared to the similarly transfected MSH2 proficient Hec59+chr2 cells (Student's t-test). Error bars represent standard errors of the mean.
B. Cell lysates from Hec59+Chr2 and Hec59 cells were analysed 48 hours after transfection, by western blotting. Antibodies directed against OGG1 and β-tubulin, were used to demonstrate sufficient reduction in expression after siRNA transfection.
C. Elevated 8-OHdG accumulation upon MSH2 deficiency and silencing of POLB, as detected by immunofluoresence. Hec59+Chr2 and Hec59 cells were transfected with either control siRNA or siRNA directed against POLB. 8-OHdG accumulation wasdetected using a fluorescein-tagged 8-OHdG binding protein by confocal microscopy. Elevated 8-OHdG immunofluoresence is associated with MSH2 deficiency and POLB silencing. DAPI staining in blue represents nuclear staining. FITC-8-OHdG in green represents 8-OHdG.
D. Increased 8-OHdG accumulation upon MLH1 deficiency and silencing of POLG. HCT116 and HCT116+chr3 cells were transfected with Control or POLG siRNA. Isolated DNA from transfected cells were analysed for 8-OHdG accumulation using a 8-OHdG ELISA assay. Oxidised lesions were quantified according to an 8-OHdG standard curve. Assays were performed in triplicate. *-p≦0.002 compared to the similarly transfected MLH1 proficient HCT116+chr3 cells (Student's t-test). Error bars represent standard errors of the mean.
E. Increased mitochondrial 8-OHdG accumulation upon MLH1 deficiency and silencing of POLG. HCT116 and HCT116+chr3 cells were transfected with Control, POLB or POLG siRNA. Nuclear and mitochondrial DNA isolated from transfected cells were analysed for 8-OHdG accumulation using a 8-OHdG ELISA assay. Oxidised lesions were quantified according to an 8-OHdG standard curve. Assays were performed in triplicate. Error bars represent standard errors of the mean.
F. Increased nuclear 8-OHdG accumulation upon MSH2 deficiency and silencing of POLB. Hec59 and Hec59+chr2 cells were transfected with Control, POLB or POLG siRNA. Nuclear and mitochondrial DNA isolated from transfected cells were analysed for 8-OHdG accumulation using a 8-OHdG ELISA assay. Oxidised lesions were quantified according to an 8-OHdG standard curve. Assays were performed in triplicate. Error bars represent standard errors of the mean.
G. Validation of nuclear and mitochondrial fractionation. HCT116, HCT116+chr3, Hec59 and Hec59+chr2 cells were transfected with Control, POLB or POLG siRNA. Nuclear and mitochondrial protein lysates were isolated from transfected cells and were analysed by western blotting. Antibodies against PCNA and cytochrome•C were used, to determine nuclear and mitochondrial fractionations, respectively.
H. POLB mRNA expression is increased after H202 treatment. POLB mRNA levels were analysed after treatment with 100 μM H202 and RNA was isolated after 15 mins, using qRT-PCR. POLB expression was normalized to that of a house-keeping gene, GAPDH. Error bars represent standard errors of the mean.

FIG. 10. OGG1 cleavage activity is decreased in the absence of POLB expression

A. Schematic model for in vitro OGG1 assay. Briefly, protein was isolated from transfected cells as indicated in FIG. 10B. The substrate is a 23 oligonucleotide containing 8-OHdG at its 11th base, labeled with 32P at its 5′ end, and annealed to its complementary strand (containing dC at the opposite base position to the 8-OHdG). Upon cleavage of the substrate by the OGG1 enzyme, the oligonucleotides were electrophoresed on a denaturing PAGE gel, followed by autoradiography.
B. Silencing of POLB expression results in the abrogation of the OGG1 mediated cleavage of 8-OHdG. HeLa cells were transfected with either control, POLB or OGG1 siRNA and incubated with a oligonucleotide substrate containing 8-OHdG, as described above. The oligonucleotides were electrophoresed and a 10 base fragment (labelled clevage product) was revealed in addition to the original 23 base oligonucleotide. Autoradiography was revealed that in the absence of POLB expression, cleavage of the 8-OHdG lesion was significantly decreased.
C. OGG1 expression is decreased upon silencing of POLB. Cell lysates from Hec59+Chr2 and Hec59 cells were analysed 72 hours after transfection with siRNA oligonucleotides, by western blotting. Antibodies directed against OGG1, POLB and β-tubulin, were used to demonstrate reduction in expression of OGG1 after transfection with POLB siRNA.

FIG. 11. POLB inhibition leads to decreased OGG1 expression via CHIP mediated degradation

A. OGG1 forms a complex with POLB. Communoprecipation assays using a POLB antibody were preformed on HeLa whole cell lysates, and analysed by western blot analysis using an antibody directed against OGG1. Autoradiography revealed an interaction between POLB and OGG1.
B. Decreased OGG1 expression after POLB silencing requires CHIP. HeLa cells were transfected with siRNA and cell lysates were analysed 72 hours later. Antibodies directed against OGG1, POLE, CHIP and β-tubulin, were used to demonstrate reduction in expression of OGG1 after transfection with POLE siRNA, which was rescued by combined silencing of POLB and CHIP.
C. Decreased OGG1 expression after POLB silencing is via proteasomal degradation. Cell lysates from HeLa cells were transfected with siRNA and after 48 hr, cells were treated with and without (50 μM) MG132. Lysates were analysed 18 hours later by western blotting. Antibodies directed against OGG1, POLE and β-tubulin, were used to demonstrate reduction in expression of OGG1 after transfection with POLB siRNA, which was rescued by treatment with the proteasomal inhibitor MG132.

FIG. 12. A model for the selective effects of BER polymerase inhibition in Mismatch Repair deficient cells Oxidised DNA lesions, including 8-OHdG can be repaired by either MMR or BER. In wild type cells, inhibition of BER by POLE or POLG silencing leads to repair of these lesions by MMR. In the absence of MSH2, POLB is essential for 8-OHdG repair. Inhibition of POLB in MSH2 deficient cells leads to the accumulation of 8-OHdG in nuclear DNA. Cells harboring these unrepaired lesions may permanently arrest or die. In cells with MLH1 deficiency, POLG inhibition leads to the accumulation of 8-OHdG in mitochondrial DNA. Again this accumulation either becomes incompatible with viability or limits the cells replicative potential.

### DETAILED DESCRIPTION

### Inhibitors

Compounds which may be employed or screened for use in the present invention for treating a DNA mismatch repair (MMR) deficient cancer, and more particularly as they are inhibitors of DNA polymerase POLP, DNA polymerase POLY, telomerase transcriptional element integrating factor (TEIF or SCYL1) and/or dihydrofolate reductase (DHFR). Some inhibitors of these polypeptides are known and further examples may be found by the application of screening technologies to these targets.

### Small Molecule Inhibitors

By way of example, to date three Polβ-specific inhibitors have been described:
Prunasin, Mizushina et al., 1999, J. Biochem., 126, 430-436.
Solanapyrone A, Mizushina et al., 2002, J. Biol. Chem., 277, 630-638.
Masticadienonic acid, Boudsocq et al., 2005, Mol., Pharmacol., 67, 1485-1492.

However, further Polβ inhibitors have be identified by the use of high throughput screening strategies (Boudsocq et al., 2005, Mol. Pharmacol., 67, 1485-1492). In this assay, DNA polymerase beta activity was determined as the amount of fluorescein-12-dCTP incorporated into a 60-mer biotinylated oligonucleotide template hybridized to a 5′ 17-mer synthetic primer. This substrate was immobilized in a streptavidin-coated combiplate C8 (ThermoLabsystem, Franklin, Mass.). The standard reaction mixture (100 μl) contained 25 mM HEPES, pH 8.5, 5 mM MgCl₂, 125 mM NaCl, 25 μmol biotinylated hybridized oligonucleotide, and 5 μg of recombinant rat Polβ in the presence of extracts or compounds. The reaction was started with the simultaneous addition of 10 μM dNTP and 1 μM fluorescein-12-dCTP. Incubation was for 150 min at 37° C., and the products were washed three times with 200 μl of 25 mM HEPES, pH 8.5, 5 mM MgCl₂, 125 mM NaCl, and 0.05%; (v/v) Tween 20. The fluorescence was measured in a Fluostar fluorimeter (BMG Labtechnologies Inc., Durham, N.C.). The HTS experiments were run on a Beckman Sagian system (Beckman Coulter, Fullerton, Calif.). Plate-handling was performed with the Optimized Robot for Chemical Analysis robotic arm (Beckman Coulter). Each positive fraction has manually controlled with the same protocol.

POLG inhibition results in depletion of mtDNA, leading to decreased synthesis of mitochondrial proteins that maintain oxidative phosphorylation pathways. Recently it has been shown that vitamin K3 (VK3; Menadione) selectively inhibits POLG. VK3 at 30 μM inhibited POLG by more than 80%, caused impairment of mitochondrial DNA replication and repair, and induced a significant increase in reactive oxygen species (ROS), leading to apoptosis (Sasaki et al., 2008). The triphosphates (TP) of many human immunodeficiency virus (HIV) nucleoside reverse transcriptase inhibitors (NRTIs); or diphosphates of phosphonate nucleotide analogs, have also been shown to inhibit POLG in vitro. Lamivudine-TP (LVD-TP), adefovir-diphosphate (ADV-DP), and tenofovir-DP (TFV-DP), as well as zalcitabine-TP (ddCTP), zidovudine-TP (AZT-TP) (Lewis et al., 1994), and other HIV antivirals, inhibit POLG activity in vitro, although the extent of inhibition varies widely (Brinkman et al., 1998; Chemington et al., 1994; Mazzucco et al., 2008),

DHFR inhibitors are also known and include:
Deaza analogs of folic acid, Kisliuk, 2003, Curr. Pharm. Des., 9(31), 2615-25.
Tomudex (D1694, raltitrexed), McGuire, Curr. Pharm. Des., 2003, 9(31), 2593-613.
Pemetrexed disodium (Eli Lilly), Norman, Curr. Opin. Investig. Drugs., 2001, November 2(11), 1611-22.
Trimetrexate, Takemura et al., Int. J. Hematol., 1997, December 66(4), 459-77.
Derivatives of tetrahydrofolate, Hartman, J. Chemother., 1993, December 5(6), 369-76.
10-Ethyl-10-deazaminopterin (10-EdAM), N10-Propargyl-5,8-dideazafolic acid (CB3717) and 5,10-Dideazatetrahydrofolic acid (DDATHF), Fry & Jackson, Cancer Metastasis Rev., 1987, 5(3), 251-70.

From McGuire (supra) antifolates are the oldest of the antimetabolite class of anticancer agents and were one of the first modern anticancer drugs. The first clinically useful antifolate, described in 1947, was 2,4-diamino-pteroylglutamate (4-amino-folic acid; aminopterin; AMT) which yielded the first ever remissions in childhood leukemia. AMT was soon superseded by its 10-methyl congener, methotrexate (MTX), based on toxicity considerations. MTX remains, with one limited exception, the only antifolate anticancer agent in clinical use to this date. Because of the safety and utility of MTX, considerable effort has been invested in attempting to design more therapeutically selective antifolates or antifolates with a wider tumor spectrum. Initially, the design was based on the burgeoning knowledge of folate-dependent pathways and the determinants of the mechanism of action of MTX. These determinants include transport, the tight-binding inhibition of its target (the folate-dependent enzyme dihydrofolate reductase (DHFR)), and metabolism of MTX to poly-γ-glutamate (Glu n) metabolites. These early studies led to the development of other antifolate DHFR inhibitors of two types: (1) “classical” analogs that use the same cellular transport systems as MTX and are also metabolized to Glun, and (2) “nonclassical” (i.e., lipophilic) analogs that do not require transport systems and that are not metabolized to Glun. Although several of these analogs have undergone clinical trial, none is proved superior to MTX.

Detailed examination of the mechanisms of cytotoxicity and selectivity of MTX showed that inhibition of both dTMP synthesis and de novo purine synthesis, secondary to DHFR inhibition, led to DNA synthesis inhibition and subsequent cell death; inhibition of other folate dependent pathways did not appear necessary for cell death. Further studies showed that the contribution of inhibition of dTMP or purine synthesis to cell death varied in different cell types. These data suggested that inhibition of one of these pathways individually might (at least in some cases) be therapeutically superior to the dual inhibition induced by MTX. Thus, in rational design and in structure-based design studies, two new classes of antifolate enzyme inhibitors were elaborated-direct inhibitors of thymidylate synthase (TMPS) and direct inhibitors of one or both of the two folate-dependent enzymes of de novo purine synthesis. Members of each class included both classical and non-classical types. After preclinical evaluation, several of these have moved into clinical trials. To date only one new TMPS inhibitor has successfully completed clinical trials and been approved for routine use; this drug, Tomudex (D1694, raltitrexed) is currently approved only in Europe and only for the treatment of colon cancer. This still represents a step forward for antifolates, however, since MTX is well-known to be ineffective in colon cancer; thus Tomudex extends the tumor range of antifolates. Antifolate development continues. Based on the immense body of knowledge now extant on antifolates, specific aspects of the mechanism of action have been the focus. Newer antifolates have been described that inhibit more than one pathway in folate metabolism, that have improved delivery, or that inhibit other targets in folate metabolism. These new analogs are in various stages of preclinical and clinical development.

The present invention also extends to the use of small molecule inhibitors found in the screening disclosed herein and to Derivatives which are compounds of similar structure and functionality to the compounds found in the high throughput screen, but with one or more modifications, are expected to have similar physiological effects to these compounds and could therefore also be of use in the treatment of MMR-deficient cancers. The screening methods of the invention may be used to screen libraries of such derivatives to optimise their activity, if necessary:

Derivatives may be designed, based on a lead compound, by modifying one or more substituents or functional groups compared to the lead compound, for example by replacing these with alternative substituents or groups which are expected to have the same or improved physiological effect. The use of derivatives having such modifications is well known to those in the art.

Accordingly, derivatives of methotrexate of use in MMR-deficient cancer treatment may include compounds of formula I, below, wherein:

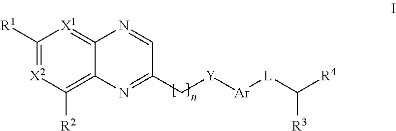

wherein X¹ and X² are N or CR⁵, where R⁵ is H, C₁₋₇ alkyl, OR^{O}, NR^{N1}R^{N2}, SR^{S}, NO₂, or halo, where R^{O}, R^{S}, R^{N1} and R^{N2} are independently H or C₁₋₇ alkyl;
R¹ and R² are each independently H, C₁₋₇ alkyl, OR^{O}, SR^{S} or NR^{N1}R^{N2}, NO₂, or halo where R^{O}, R^{S}, R^{N1} and R^{N2} are as previously defined;
Y is O, S, NR^{N1}, or CR⁶R⁷, where R⁶ and R⁷ are independently H, C₁₋₇ alkyl, or halo, and R^{O}, and R^{N1} are as previously defined;
Ar is a C₅₋₂₀ aromatic ring optionally substituted with one or more R^{S}, where R^{S} is as previously defined;
L is a linker selected from C(═O), C(═O)O and C′(═O)NR^{N1} where R^{N1} is as previously defined;
n is from 0 to 3;
R³ and R⁴ are independently (CH₂)ₘCO₂R^{O} where m is from 0 to 5 and R^{O} is as previously defined.

Preferably, at least one of X¹ and X² is N. More preferably, both are N. If X¹ or X² are CR⁵, preferably R⁵ is H, halo or C₁₋₄ alkyl, most preferably H.

Preferably, R¹ and R² are H, C₁₋₄ alkyl or halo, most preferably H.

Preferably n is 1.

Preferably, Y is NR^{N1} where R^{N1} is preferably C₁₋₄ alkyl, most preferably Me.

Preferably, L is an amide linker C(═O)NR^{N1} where R^{N1} is preferably H or Me.

Ar is preferably, a benzene or thiophene ring.

R³ and R⁴ are each preferably (CH₂)ₘCO₂H. For R³ m is preferably 0. For R⁴ m is preferably 2.

Derivatives of parthenolide may include compounds of formula II:

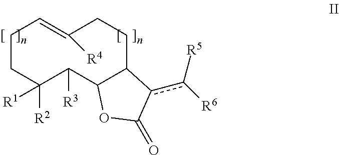

wherein R¹, R², and R³ are each independently H, OR^{O}, SR^{S} or NR^{N1}R^{N2}, where R^{O}, R^{S}, R^{N1} and R^{N2} are independently H or C₁₋₇ alkyl, or R² and R³ together with the carbon atoms to which they are bound form a C₃₋₅ carbocyclic or heterocyclic ring;
R⁴ is H or C₁₋₇ alkyl;
R⁵ and R⁶ are each independently H, C₁₋₇ alkyl, OR^{O}, SR^{S} or NR^{N3}R^{N4}, where R^{N3} and R^{N4} are independently H, C₁₋₇ alkyl, and C₅₋₂₀ aryl, where each aryl or alkyl group is optionally substituted by OR^{O}, SR^{S}, NR^{N1}R^{N2}, C₁₋₇ alkyl or halo, and R^{O}, R^{s}R^{N1} and R^{N2} are as previously defined;
n is from 0 to 3;
and the dashed line indicates an optional double bond.

R¹ and R⁴ are preferably H or C₁₋₄ alkyl, and are most preferably methyl.

Preferably R² and R³ are linked to form a 3-5 membered ring which preferably contains an oxygen atom. Most preferably R² and R³ together with the carbons to which they are bound form an epoxide ring.

Preferably, at least one of R⁵ and R⁶ is H.

If the optional double bond is not present, one of R⁵ or R⁶ is preferably NR^{N3}R^{N4}.

Each n is preferably 1.

Derivatives of menadione may include compounds of formula III:

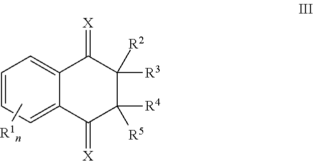

wherein X¹ and X² are independently O or NR^{N1}, where R^{N1} is H or C₁₋₇ alkyl;
each R¹ is a substituent on the phenyl ring and is selected from halo, NO₂, C₁₋₇ alkyl, C₅₋₂₀ aryl, OR^{O}, SR^{S} or NR^{N1}R^{N2}, or two R¹ together with the atoms to which they are bound may form an alicyclic or aromatic ring fused to the phenyl ring, wherein R^{O}, R^{S}, R^{N1} and R^{N2} are independently H or C₁₋₇ alkyl;
R², R³, R⁴ and R⁵ are each independently H, C₁₋₇ alkyl, C₅₋₂₀ aryl, OR^{O}, SR^{S}, NR^{N1}R^{N2}, or halo;
or R³ and R⁴ together form a n bond between the carbon atoms to which they are bound, or R³ and R⁴, together with the carbon atoms to which they are bound form an optionally substituted C₃₋₆ carbocyclic or heterocyclic ring, and R² and R⁵ are as previously defined;
n is from 0 to 4;
and where each aryl or alkyl group is optionally substituted by OR^{O}, SR^{S}, NR^{N1}R^{N2}, C₁₋₇ alkyl or halo.

Preferably at least one of X² and X² is O. Most preferably both are O.

R¹ is preferably OR^{O}, or two R¹ together with the atoms to which they are bound form an alicyclic or aromatic ring fused to the phenyl ring. More preferably they form a fused lactone ring.

Preferably R³ and R⁴ together with the carbon atoms to which they are bound form a ring. More preferably the ring is an epoxide or a lactone-containing ring.

Alternatively, R³ and R⁴ preferably form a it bond between the carbons to which they are attached.

R² is preferably H or C₁₋₄ alkyl, most preferably H or Me.

R⁵ is preferably H, Me or OR^{O}.

### DEFINITIONS

C₁₋₇alkyl: The term “C₁₋₇alkyl”, as used herein, pertains to a monovalent moiety obtained by removing a hydrogen atom from a C₁₋₇ hydrocarbon compound having from 1 to 7 carbon atoms, which may be aliphatic or alicyclic, or a combination thereof, and which may be saturated, partially unsaturated, or fully unsaturated. Corresponding terms such as “C₁₋₄ alkyl” pertain to a moiety so obtained from a hydrocarbon having from 1 to 4 carbon atoms, and so on.

Examples of saturated linear C₁₋₇ alkyl groups include, but are not limited to, methyl, ethyl, n-propyl, n-butyl, and n-pentyl (amyl).

Examples of saturated branched C₁₋₇ alkyl groups include, but are not limited to, iso-propyl, iso-butyl, sec-butyl, tert-butyl, and neo-pentyl.

Examples of saturated alicyclic C₁₋₇ alkyl groups (also referred to as “C₃₋₇ cycloalkyl” groups) include, but are not limited to, groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of unsaturated C₁₋₇ alkyl groups which have one or more carbon-carbon double bonds (also referred to as “C₂₋₇ alkenyl” groups) include, but are not limited to, ethenyl (vinyl, —CH═CH₂), 2-propenyl (allyl, —CH—CH═CH₂), isopropenyl (—C(CH₂)═CH₂), butenyl, pentenyl, and hexenyl.

Examples of unsaturated C₁₋₇alkyl groups which have one or more carbon-carbon triple bonds (also referred to as “C₂₋₇ alkynyl” groups) include, but are not limited to, ethynyl (ethinyl) and 2-propynyl (propargyl).

Examples of unsaturated alicyclic (carbocyclic) C₁₋₇ alkyl groups which have one or more carbon-carbon double bonds (also referred to as “C₂₋₇cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

C₃₋₂₀ heterocyclyl: The term “C₃₋₂₀ heterocyclyl”, as used herein, pertains to a monovalent moiety obtained by removing a hydrogen atom from a ring atom of a C₃₋₂₀ heterocyclic compound, said compound having one ring, or two or more rings (e.g., spiro, fused, bridged), and having from 3 to 20 ring atoms, atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms. “C₂₋₂₀” denotes ring atoms, whether carbon atoms or heteroatoms. The term “C₃₋₂₀ heterocyclic ring” may also be used and should be construed accordingly; this may refer to a multivalent moiety. Similarly the term “C₃₋₂₀ alicyclic ring” may be used for rings not containing heteroatoms.

Examples of C₃₋₂₀ heterocyclyl groups having one nitrogen ring atom include, but are not limited to, those derived from aziridine, azetidine, pyrrolidines (tetrahydropyrrole), pyrroline (e.g., 3-pyrroline, 2,5-dihydropyrrole), 2H-pyrrole or 3H-pyrrole (isopyrrole, isoazole), piperidine, dihydropyridine, tetrahydropyridine, and azepine.

Examples of C₃₋₂₀ heterocyclyl groups having one oxygen ring atom include, but are not limited to, those derived from oxirane, oxetane, oxolane (tetrahydrofuran), oxole (dihydrofuran), oxane (tetrahydropyran), dihydropyran, pyran (C₆), and oxepin. Examples of substituted C₂₋₂₀ heterocyclyl groups include sugars, in cyclic form, for example, furanoses and pyranoses, including, for example, ribose, lyxose, xylose, galactose, sucrose, fructose, and arabinose.

Examples of C₃₋₂₀ heterocyclyl groups having one sulphur ring atom include, but are not limited to, those derived from thiirane, thietane, thiolane (tetrahydrothiophene), thiane (tetrahydrothiopyran), and thiepane.

Examples of C₃₋₂₆ heterocyclyl groups having two oxygen ring atoms include, but are not limited to, those derived from dioxolane, dioxane, and dioxepane.

Examples of C₃₋₂₀ heterocyclyl groups having two nitrogen ring atoms include, but are not limited to, those derived from imidazolidine, pyrazolidine (diazolidine), imidazoline, pyrazoline (dihydropyrazole), and piperazine.

Examples of C₃₋₂₀ heterocyclyl groups having one nitrogen ring atom and one oxygen ring atom include, but are not limited to, those derived from tetrahydrooxazole, dihydrooxazole, tetrahydroisoxazole, dihydroisoxazole, morpholine, tetrahydrooxazine, dihydrooxazine, and oxazine.

Examples of C₃₋₂₀ heterocyclyl groups having one oxygen ring atom and one sulphur ring atom include, but are not limited to, those derived from oxathiolane and oxathiane (thioxane).

Examples of C₃₋₂₀ heterocyclyl groups having one nitrogen ring atom and one sulphur ring atom include, but are not limited to, those derived from thiazoline; thiazolidine, and thiomorpholine.

Other examples of C₃₋₂₀ heterocyclyl groups include, but are not limited to, oxadiazine and oxathiazine.

Examples of heterocyclyl groups which additionally bear one or more oxo (═O) groups, include, but are not limited to, those derived from:

C₅ heterocyclics, such as furanone, pyrone, pyrrolidone (pyrrolidinone), pyrazolone (pyrazolinone), imidazolidone, thiazolone, and isothiazolone;
C₆ heterocyclics, such as piperidinone (piperidone), piperidinedione, piperazinone, piperazinedione, pyridazinone, and pyrimidinone (e.g., cytosine, thymine, uracil), and barbituric acid;
fused heterocyclics, such as oxindole, purinone (e.g., guanine), benzoxazolinone, benzopyrone (e.g., coumarin); cyclic anhydrides (—C(═O)—O—C(═O)— in a ring), including but not limited to maleic anhydride, succinic anhydride, and glutaric anhydride;
cyclic carbonates (—O—C(═O)—O— in a ring), such as ethylene carbonate and 1,2-propylene carbonate;
imides (—C(═O)—NR—C(═O)— in a ring), including but not limited to, succinimide, maleimide, phthalimide, and glutarimide; lactones (cyclic esters, —O—C(═O)— in a ring), including, but not limited to, β-propiolactone, γ-butyrolactone, δ-valerolactone (2-piperidone), and ε-daprolactone;
lactams (cyclic amides, —NR—C(═O)— in a ring), including, but not limited to, β-propiolactam, γ-butyrolactam (2-pyrrolidone), δ-valerolactam, and ε-caprolactam;
cyclic carbamates (—O—C(═O)—NR— in a ring), such as 2-oxazolidone;
cyclic ureas (—NR—C(═O)—NR— in a ring), such as 2-imidazolidone and pyrimidine-2,4-dione (e.g., thymine, uracil).

C₅₋₂₀aryl: The term “C₅₋₂₀ aryl”, as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms. The term “C₅₋₂₀ aromatic ring” may also be used and should be construed accordingly; this may refer to a multivalent moiety.

The ring atoms may be all carbon atoms, as in “carboaryl groups”, in which case the group may conveniently be referred to as aC “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), naphthacene (C₁₈), and pyrene (C₁₆).

Examples of aryl groups which comprise fused rings, one of which is not an aromatic ring, include, but are not limited to, groups derived from indene and fluorene.

Alternatively, the ring atoms may include one or more heteroatoms, including but not limited to oxygen, nitrogen, and sulphur, as in “heteroaryl groups”. In this case, the group may conveniently be referred to as a “C₅₋₂₀heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

Examples of C₅₋₂₀heteroaryl groups which comprise fused rings, include, but are not limited to, C₉ heterocyclic groups derived from benzofuran, isobenzofuran, indole, isoindole, purine (e.g., adenine, guanine), benzothiophene, benzimidazole; C₁₀ heterocyclic groups derived from quinoline, isoquinoline, benzodiazine, pyridopyridine, quinoxaline; C₁₃ heterocyclic groups derived from carbazole, dibenzothiophene, dibenzofuran; C₁₄ heterocyclic groups derived from acridine, xanthene, phenoxathiin, phenazine, phenoxazine, phenothiazine.

The term ‘halo’ refers to —F, —Cl, —Br, and —I substituents. Fluoro (—F) and chloro (—Cl) substituents are usually preferred.

The above C₁₋₇ alkyl, C₃₋₂₀ heterocyclyl and C₅₋₂₀ aryl groups, whether alone or part of another substituent, may themselves optionally be substituted with one or more groups selected from themselves and the additional substituents listed below.

### Halo: —F, —Cl, —Br, and —I.

### Hydroxy: —OH.

Ether: —OR, wherein R is an ether substituent, for example, a C₁₋₇ alkyl group (also referred to as a C₁₋₇alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

C₁₋₇ alkoxy: —OR, wherein R is a C₁₋₇ alkyl group. Examples of C₁₋₇ alkoxy groups include, but are not limited to, —OCH₃ (methoxy), —OCH₂CH₃ (ethoxy) and —OC(CH₃)₃ (tert-butoxy).

Oxo (keto, -one): ═O. Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam (2-pyrrolidone), δ-valerolactam, and ε-caprolactam.

Imino (imine): ═NR, wherein R is an imino substituent, for example, hydrogen, C₁₋₇ alkyl group, a C₃₋₂₀heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇alkyl group (also referred to as C₁₋₇alkylacyl or C₁₋₇ alkanoyl), a C₃₋₂₀ heterocyclyl group (also referred to as C₃₋₂₀ heterocyclylacyl); or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably hydrogen or a alkyl group, and R² is an acyl substituent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph.

Acylureido: —N(R¹)C(O)NR²C(O)R³ wherein R¹ and R² are independently ureido substituents, for example, hydrogen, a C₁₋₇ alkyl group, a C₃₋₂₀ heterocyclyl group, or a C₅₋₂₀ aryl group, preferably hydrogen or a C₂₋₂₀ alkyl group. R³ is an acyl group as defined for acyl groups. Examples of acylureido groups include, but are not limited to, —NHCONHC(O)H, —NHCONMeC(O)H, —NHCONEtC(O)H, —NHCONMeC(O)Me, —NHCONEtC(O)Et, —NMeCONHC(O)Et, —NMeCONHC(O)Me, —NMeCONHC(O)Et, —NMeCONMeC(O)Me, —NMeCONEtC(O)Et, and —NMeCONHC(O)Ph.

Carbamate: —NR¹—C(O)—OR² wherein R¹ is an amino substituent as defined for amino groups and R² is an ester group as defined for ester groups. Examples of carbamate groups include, but are not limited to, —NH—C(O)—O-Me, —NMe-C(O)—O-Me, —NH—C(O)—O-Et, —NMe-C(O)—O-t-butyl, and —NH—C(O)—O-Ph.

Thioamido (thiocarbamyl): —C(═S)NR¹R², wherein R¹ and R² are independently amino substituents, as defined for amino groups. Examples of amido group's include, but are not limited to, —C(═S)NH₂, —C(═S)NHCH₃, —C(═S)N(CH₃)₂, and —C(═S)NHCH₂CH₃.

Tetrazolyl: a five membered aromatic ring having four nitrogen atoms and one carbon atom,

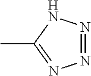

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHC(CH₂)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇ alkyl group.

Amidine: —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a alkyl group. An example of an amidine group is —C(═NH)NH₂.

Carbazoyl (hydrazinocarbonyl): —C(O)—NN—R¹ wherein R¹ is an amino substituent as defined for amino groups. Examples of azino groups include, but are not limited to, —C(O)—NN—H, —C(O)—NN-Me, —C(O)—NN-Et, —C(O)—NN-Ph, and —C(O)—NN—CH₂-Ph.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₃₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃, —S(═O)₂C₄F₉ (nonaflyl), —S(═O)₂CH₂CF₃ (tresyl), —S(═O)₂Ph (phenylsulfonyl), 4-methylphenylsulfonyl (tosyl), 4-bromophenylsulfonyl (brosyl), and 4-nitrophenyl (nosyl).

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfonyloxy: —OS(═O)₂R, wherein R is a sulfonyloxy substituent, for example, a alkyl group, a C₂₋₂₀ heterocyclyl group, or a C₅₋₂₀ aryl group, preferably a C₁₋₇ alkyl group. Examples of sulfonyloxy groups include, but are not limited to, —OS(═O)₂CH₃ and —OS(═O)₂CH₂CH₃

Sulfinyloxy: —OS(═O)R, wherein R is a sulfinyloxy substituent, for example, a alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfinamino: —NR¹S(═O) R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇alkyl group. Examples of sulfinamino groups include, but are not limited to, —NHS(═O)CH₃ and —N(CH₃) S(═O)C₆H₅

Sulfamyl: —S(═O)NR¹R², wherein R¹ and R² are independently amino substituents, as defined for amino groups. Examples of sulfamyl groups include, but are not limited to, —S(═O)NH₂, —S(═O) NH(CH₃), —S(═O)N(CH₃)₂, —S(═O)NH(CH₂CH₃) (═O)N(CH₂CH₃)₂, and —S(═O)NHPh.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅. A special class of sulfonamino groups are those derived from sultams—in these groups one of R¹ and R is a C₅₋₂₀ aryl group, preferably phenyl, whilst the other of R¹ and R is a bidentate group which links to the C₅₋₂₀ aryl group, such as a bidentate group derived from a C₁₋₇ alkyl group.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H; a alkyl group, or a C₅₋₂₀ aryl group. Examples of phosphoramidite groups include, but are not limited to, —OP(OCH₂CH₃)—N(CH₃)₂, —OP(OCH₂CH₂)—N(i-Pr)₂, and —OP(OCH₂CH₂CN)—N(i-Pr)_{2.}

Phosphoramidate: —OP(═O)(OR²)—NR²₂, where R¹ and R² are phosphoramidate substituents, for example, —H, a (optionally substituted) C₁₋₇ alkyl group, a C₃₋₂₀ heterocyclyl group, or a Cₛ₋₂₀ aryl group, preferably —H, a alkyl group, or a C₅₋₂₀ aryl group. Examples of phosphoramidate groups include, but are not limited to, —OP(═O) (OCH₂CH₃)—N(CH₃)₂, —OP(═O)(OCH₂CH₃)—N(i-Pr)₂, and —OP(═O) (OCH₂CH₂CN)—N(i-Pr)₂.

In many cases, substituents may themselves be substituted. For example, a C₁₋₇ alkoxy group may be substituted with, for example, a C₁₋₇ alkyl (also referred to as a C₁₋₇ alkyl-C₁₋₇ alkoxy group), for example, cyclohexylmethoxy, a C₃₋₂₀ heterocyclyl group (also referred to as a C₅₋₂₀ aryl-C₁₋₇ alkoxy group), for example phthalimidoethoxy, or a C₅₋₂₀ aryl group (also referred to as a C₅₋₂₀aryl-C₁₋₇alkoxy group), for example, benzyloxy.

### Antibodies

Antibodies may be employed in the present invention as an example of a class of inhibitor useful for treating a DNA mismatch repair (MMR) deficient cancer, and more particularly as inhibitors of DNA polymerase POLβ, DNA polymerase POLY, telomerase transcriptional element integrating factor (TEIF or SCYL1) and/or dihydrofolate reductase (DHFR). They may also be used in the methods disclosed herein for assessing an individual having cancer or predicting the response of an individual having cancer, in particular for determining whether the individual has a DNA mismatch repair deficient cancer that might be treatable according to the present invention.

As used herein, the term “antibody” includes an immunoglobulin whether natural or partly or wholly synthetically produced. The term also covers any polypeptide or protein comprising an antibody binding domain. Antibody fragments which comprise an antigen binding domain are such as Fab, scFv, Fv, dAb, Fd; and diabodies. It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2,188,638 A or EP 0 239 400 A.

Antibodies can be modified in a number of ways and the term “antibody molecule” should be construed as covering any specific binding member or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP 0 120 694 A and EP 0 125 023 A.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242; 423-426, 1988; Huston et al, PNAS USA, 85: 5879-5883, 1958); (viii) bispecific single chain Fv dimers (WO 93/11161) and (ix) “diabodies”, multivalent or multispecific fragments constructed by gene fusion (WO 94/13804; Holliger et al, P.N.A.S. USA, 90: 6444-6448, 1993); (x) immunoadhesins (WO 98/50431). Fv, scFv or diabody molecules may be stabilised by the incorporation of disulphide bridges linking the VH and VL domains (Reiter et al, Nature Biotech, 14: 1239-1245, 1996). Minibodies comprising a scFv joined to a CH3 domain may also be made (Hu et al, Cancer Res., 56: 3055-3061, 1996).

Preferred antibodies used in accordance with the present invention are isolated, in the sense of being free from contaminants such as antibodies able to bind other polypeptides and/or free of serum components. Monoclonal antibodies are preferred for some purposes, though polyclonal antibodies are within the scope of the present invention.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid. Antibodies may modulate the activity of the polypeptide to which they bind and so, if that polypeptide has a deleterious effect in an individual, may be useful in a therapeutic context (which may include prophylaxis).

### Peptide Fragments

Another class of inhibitors useful for treating a DNA mismatch repair (MMR) deficient cancer includes peptide fragments that interfere with the activity of DNA polymerase β, DNA polymerase POLγ, TEIF or DHFR. Peptide fragments may be generated wholly or partly by chemical synthesis that block the catalytic sites of DNA polymerase β, TEIF or DHFR. Peptide fragments can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Other candidate compounds for inhibiting DNA polymerase β, TEIF or DHFR, may be based on modelling the 3-dimensional structure of these enzymes and using rational drug design to provide candidate compounds with particular molecular shape, size and charge characteristics. A candidate inhibitor, for example, may be a “functional analogue” of a peptide fragment or other compound which inhibits the component. A functional analogue has the same functional activity as the peptide or other compound in question. Examples of such analogues include chemical compounds which are modelled to resemble the three dimensional structure of the component in an area which contacts another component, and in particular the arrangement of the key amino acid residues as they appear.

### Nucleic Acid Inhibitors

Another class of inhibitors useful for treatment of a DNA mismatch repair (MMR) deficient cancer includes nucleic acid inhibitors of DNA polymerase POLβ(NM_{—}002690.1), DNA polymerase POLγ (NM_{—}001126131.1), telomerase transcriptional element integrating factor (TEIF or SCYL1)(NM_{—}001048218.1 and NM_{—}020680.3) or dihydrofolate reductase (DHFR) (NM_{—}000791.3), or the complements thereof, which inhibit activity or function by down-regulating production of active polypeptide. This can be monitored using conventional methods well known in the art, for example by screening using real time PCR as described in the examples.

Expression of DNA polymerase POLβ, DNA polymerase POLy, telomerase transcriptional element integrating factor (TEIF or SCYL1) and/or dihydrofolate reductase (DHFR) may be inhibited using anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example in Peyman & Ulman, Chemical Reviews, 90:543-584, 1990 and Crooke, Ann. Rev. Pharmacol. Toxicol., 32:329-376, 1992.

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g., about 15, 16 or 17 nucleotides.

An alternative to anti-sense is to use a copy of all or part of the target gene inserted in sense, that is the same, orientation as the target gene, to achieve reduction in expression of the target gene by co-suppression (Angell & Baulcombe, The EMBO Journal 16(12):3675-3684, 1997 and Voinnet & Baulcombe, Nature, 389: 553, 1997). Double stranded RNA (dsRNA) has been found to be even more effective in gene silencing, than both sense or antisense strands alone (Fire et al, Nature 391, 806-811, 1998). dsRNA mediated silencing is gene specific and is often termed RNA interference (RNAi). Methods relating to the use of RNAi to silence genes in C. elegans, Drosophila, plants, and mammals are known in the art (Fire, Trends Genet., 15: 358-363, 1999; Sharp, RNA interference, Genes Dev. 15: 485-490 2001; Hammond et al., Nature Rev. Genet. 2: 110-1119, 2001; Tuschl, Chem. Biochem. 2: 239-245, 2001; Hamilton et al., Science 286: 950-952, 1999; Hammond, et al., Nature 404: 293-296, 2000; Zamore et al., Cell, 101: 25-33, 2000; Bernstein, Nature, 409: 363-366, 2001; Elbashir et al, Genes Dev., 15: 188-200, 2001; WO01/29058; WO99/32619, and Elbashir et al, Nature, 411: 494-498, 2001).

RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore, Nature Structural Biology, 8, 9, 746-750, 2001.

RNAi may also be efficiently induced using chemically synthesized siRNA duplexes of the same structure with 3′-overhang ends (Zamore et al, Cell, 101: 25-33, 2000). Synthetic siRNA duplexes have been shown to specifically suppress expression of endogenous and heterologeous genes in a wide range of mammalian cell lines (Elbashir et al, Nature, 411: 494-498, 2001).

Another possibility is that nucleic acid is used which on transcription produces a ribozyme, able to cut nucleic acid at a specific site and therefore also useful in influencing gene expression, e.g., see Kashani-Sabet & Scanlon, Cancer Gene Therapy, 2(3): 213-223, 1995 and Mercola & Cohen, Cancer Gene Therapy, 2(1): 47-59, 1995.

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAS” (siRNAs) or “microRNAs” (miRNAs) depending on their origin. Both types of sequence may be used to down-regulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g, of one or two (ribo)nucleotides, typically a UU of dTdT 3′ overhang. Based on the disclosure provided herein, the skilled person can readily design suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression-systems (e.g. vectors). In a preferred embodiment the siRNA is synthesized synthetically.

Longer double stranded RNAs may be processed in the cell to produce siRNAs (e.g. see Myers, Nature Biotechnology, 21: 324-328, 2003). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17: 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

Alternatively, siRNA molecules may be synthesized using standard solid or solution phase synthesis techniques, which are known in the art. Linkages between nucleotides may be phosphodiester bonds or alternatives, e.g., linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules, which are more, or less, stable than unmodified siRNA.

The term ‘modified nucleotide base’ encompasses nucleotides with a covalently modified base and/or sugar. For example, modified nucleotides include nucleotides having sugars, which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; or 2; azido-ribose, carbocyclic sugar analogues a-anomeric sugars; epimeric sugar such as arabinose, xyloses or lyxoses, pyranose sugars, furanose sugars and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N-6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N-6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine.

### Methods of Screening

In some aspects, the present invention is concerned with methods of screening candidate compounds to determine whether one or more candidate agents are likely to be useful for the treatment of MMR-deficient cancer. As described herein, there are three preferred general approaches that may be used for these methods of screening, either alone or in any combination or order.

In a first approach, a method of screening may involve using cell lines to determine whether a candidate agent is synthetically lethal in a first cell line which is deficient for a component of the DNA mismatch repair. (MMR) pathway. This method preferably also uses a second cell line that is proficient for said component of the DNA mismatch repair (MMR) pathway as a control and candidate agents are selected which are synthetically lethal in the first cell line and which preferably do not cause any substantial amount cell death in the second cell line and/or normal cells. Thus, in this embodiment of the invention exploits synthetic lethality in cancer cells. Two mutations are synthetically lethal if cells with either of the single mutations are viable, but cells with both mutations are inviable. Identifying synthetic lethal combinations therefore allows a distinct approach to identifying therapeutic targets that allows selective killing of tumour cells. Preferably, the method is carried out using cancer cell lines, e.g. mammalian or human cancer cell lines, and more preferably MSH2-, MLH1-, MSH6-, PMS1 or PMS2-deficient cancer cell lines.

One preferred way of initially identifying synthetic lethal interactions involves the use of RNAi screens. Synthetic lethality describes the scenario in which two normally non-essential genes become essential when both are lost, or inhibited. Targeting a gene that is synthetically lethal with a cancer specific mutation should selectively kill tumour cells while sparing normal cells. One of the major advantages of this approach is the ability to target cancer cells containing loss-of-function mutations, that is, mutations in tumour suppressor genes. Previously, it has been difficult to devise therapeutic strategies to target these mutations as recapitulating tumour suppressor function is technically difficult. Most pharmacological agents inhibit rather than activate protein function and therefore cannot be used to target loss-of-function alterations in tumours. Identification of synthetic lethal relationships with tumour suppressor genes could allow cells that contain the tumour suppressor mutations to be selectively killed.

The use of synthetic lethality to target cancer-specific mutations has been demonstrated by the selective killing of cells with breast cancer (BRCA) gene defects using poly(ADP ribose) polymerase (PARP) inhibitors. These inhibitors showed profound selectivity, killing cells with BRCA1 or BRCA2 deficiency, while normal cells were unaffected: Inhibition of PARP leads to the persistence of DNA lesions that cannot be repaired in BRCA-deficient cells, which have a defect in DNA repair, but can be processed in normal cells. In this BRCA and PARP example, the synthetic lethal targets were combined on the basis of known mechanisms of action, but more generally and in the present work synthetic lethal targets cannot be rationally identified in this manner. However, with the advent of high-throughput RNAi screens it is now possible, in principle, to perform large-scale synthetic-lethal gene identification in mammalian cells, as is routinely done in yeast. Screening deletion mutants that have defects in cell-cycle checkpoint or DNA repair mechanisms in yeast has yielded synthetically lethal genes and small-molecule inhibitors. Using mammalian isogenic-paired cell lines that differ in a single genetic target, RNAi can be used to identify drug targets that when inhibited will result in the selective death of tumour cells.

Chemical screens have been performed previously on isogenic cancer cell lines for synthetic lethal interactions. However, such approaches have the significant disadvantage of having to identify the cellular targets of an active small molecule. This can be achieved by illustrating the affinity of a small molecule for a particular protein, but this is time-consuming and suffers the limitation that irrelevant proteins will bind in addition to the target. A variation on the synthetic lethality theme is to use compounds that inhibit a cancer-specific target and then screen RNAi libraries to identify targets that selectively kill the cells treated with this compound.

Alternatively or additionally, a second method of screening may be employed based on the work described herein in which protein targets are identified as being synthetically lethal when their expression is inhibited in MMR-deficient cancers. These protein targets include DNA polymerase POLP, DNA polymerase POLY, telomerase transcriptional element integrating factor (TEIF or SCYL1) and/or dihydrofolate reductase (DHFR). Accordingly, methods of screening may be carried out for identifying candidate agents that are capable of inhibiting an activity of one or more of these targets, for subsequent use of development as agents for the treatment of MMR-deficient cancer. Conveniently, this may be done in an assay buffer to help the components of the assay interact, and in a multiple well format to test a plurality of candidate agents.

Alternatively or additionally, a third method of screening may be used based on the results disclosed herein that demonstrate that accumulation of 8-OHdG lesions in cancer cell line, leading to cell death. Accordingly, a method of screening candidate compounds based on these findings can be used, for example in which a cell line deficient in the component of the DNA mismatch repair (MMR) pathway is contacted with a candidate agents to determine whether the candidate agent causes 8-OHdG to accumulate in the cell line. 8-OHdG accumulation can easily be determined using techniques well known in the art, such as ELISA assays. These assays are commercially available from Cell Biolabs, Inc, San Diego, USA. Generally, the accumulation of 8-OHdG in cancer cells may be determined using assays well known in the art, such as the ELISA assay and the formation of lesions is associated with an increase in the level of 8-OHdG in the cancer cells, for example as compared to the basal level caused by the normal metabolism of the cell.

By way of example, the candidate agent may be a known inhibitor of one of the protein targets disclosed herein, an antibody, a peptide, a nucleic acid molecule or an organic or inorganic compound, e.g. molecular weight of less than 100 Da. In some instances the use of candidate agents that are compounds is preferred. However, for any type of candidate agent, combinatorial library technology provides an efficient way of testing a potentially vast number of different substances for ability to modulate activity of a target protein. Such libraries and their use are known in the art. The present invention also specifically envisages screening candidate agents known for the treatment of other conditions, and especially other forms of cancer, i.e. non-MMR deficient cancer. This has the advantage that the patient or disease profile of known therapeutic agents might be expanded or modified using the screening techniques disclosed herein, or for therapeutic agents in development, patient or disease profiles established that are relevant for the treatment of MMR-deficient cancer.

Following identification of a candidate agent for further investigation, the agent in question may be tested to determine whether it is not lethal to normal cells or otherwise is suited to therapeutic use. Following these studies, the agent may be manufactured and/or used in the preparation of a medicament, pharmaceutical composition or dosage form.

The development of lead agents or compounds from an initial hit in screening assays might be desirable where the agent in question is difficult or expensive to synthesise or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process. In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetics found by this approach can then be screened to see whether they have the target property, or to what extent they exhibit it. Further optimisation or modification can then be carried out to arrive at one or more final mimetics for in vivo or clinical testing.

### Treatment of Cancer

The present invention provides methods and medical uses for the treatment of DNA mismatch repair (MMR) pathway deficient cancer.

The MMR-deficient cancer may arise because an individual has a mutation in a gene in the DNA mismatch repair pathway, and especially in the context of the present invention one or more mutations in the MSH2 and/or MLH1 and/or MSH6 and/or PSM1 and/or PSM2 genes. By way of example, a review of mutations in the hMSH2 and hMLH1 genes linked to the occurrence of colorectal cancer is provided in Mitchell et al, Am. J. Epidemiol., 156: 885-902, 2002. Examples of forms of MMR deficient cancer include cancer with a MLH1 or MSH2 deficient phenotypes. These include colorectal cancers in which loss of the MMR pathway is observed in 10-15% of sporadic colorectal cancers, often as a result of aberrant MLH1 promoter methylation, and forms of colorectal cancer in which germline mutations in the MMR genes MLH1 and MSH2 predisposes individuals to hereditary non-polyposis colorectal cancer (HNPCC), also known as Lynch syndrome. Individuals with mutations in the MLH1 or MSH2 genes are also susceptible to extra-colonic tumours such as endometrial, stomach, and transitional cell carcinoma of the urinary tract. Other individuals, for example those having biallelic mutations in the MMR genes, have been associated with childhood onset of hematological and brain malignancies. Mutations in MSH2 and MLH1 are associated with Muir-Torre Syndrome, a rare autosomal dominant genodermatosis, which predisposes to visceral malignancies and sebaceous gland. Recently, the presence of p53 and MSH2 mutations in hepatocellular carcinoma patients has been suggested as an indicator of poor survival.

The systematic genetic investigation of HNPCC has also led to the identification of other the DNA-mismatch repair (MMR) genes as constituting a major pathway to colorectal cancer in syndromic cases. Thus, HNPCC has also been shown to be caused by germline mutations in the PMS1, PMS2 and MSH6 genes, although the contribution of mutations in these genes is thought to be less significant than for MLH1 and MSH2, see for example Akiyama et al, 1997; Miyaki et al.; 1997; and Peltomaki, 2005.

In other embodiments, the MMR-deficient cancer is characterised by the cancer cells having a defect in DNA mismatch repair or the cancer cells exhibiting epigenetic inactivation of a gene in the MMR pathway, or loss of the loss of protein function. In these embodiments of the present invention, preferably the gene in the MMR pathway is MSH2, MLH1, MSH6, PMS1 or PMS2.

More generally, a cancer may be identified as a MMR-deficient cancer by determining the activity of a component of the MMR pathway in a sample of cells from an individual. The sample may be of normal cells from the individual where the individual has a mutation in a gene in the MMR pathway or the sample may be of cancer cells, e.g. where the cells forming a tumour exhibit defects in DNA mismatch repair. Activity may be determined relative to a control, for example in the case of defects in cancer cells, relative to non-cancerous cells, preferably from the same tissue. The activity of the MMR pathway may be determined by using techniques well known in the art such as Western blot analysis, immunohistology, chromosomal abnormalities, enzymatic or DNA binding assays and plasmid-based assays.

In some embodiments, a cancer may be identified as a MMR-deficient cancer by determining the presence in a cell sample from the individual of one or more variations, for example, polymorphisms or mutations, in a nucleic acid encoding a polypeptide which is a component of the MMR pathway.

Sequence variations such as mutations and polymorphisms may include a deletion, insertion or substitution of one or more nucleotides, relative to the wild-type nucleotide sequence. The one or more variations may be in a coding or non-coding region of the nucleic acid sequence and, may reduce or abolish the expression or function of the MMR pathway. In other words, the variant nucleic acid may encode a variant polypeptide which has reduced or abolished activity or may encode a wild-type polypeptide which has little or no expression within the cell, for example through the altered activity of a regulatory element. A variant nucleic acid may have one or more mutations or polymorphisms relative to the wild-type sequence.

The presence of one or more variations in a nucleic acid which encodes a component of the MMR pathway, may be determined by detecting, in one or more cells of a test sample, the presence of an encoding nucleic acid sequence which comprises the one or more mutations or polymorphisms, or by detecting the presence of the variant component polypeptide which is encoded by the nucleic acid sequence.

Various methods are available for determining the presence or absence in a sample obtained from an individual of a particular nucleic acid sequence, for example a nucleic acid sequence, which has a mutation or polymorphism that reduces or abrogates the expression or activity of a MMR pathway component. Furthermore, having sequenced nucleic acid of an individual or sample, the sequence information can be retained and subsequently searched without recourse to the original nucleic acid itself. Thus, for example, scanning a database of sequence information using sequence analysis software may identify a sequence alteration or mutation.

Methods according to some aspects of the present invention may comprise determining the binding of an oligonucleotide probe to nucleic acid obtained from the sample, for example, genomic DNA, RNA or cDNA. The probe may comprise a nucleotide sequence which binds specifically to a nucleic acid sequence which contains one or more mutations or polymorphisms and does not bind specifically to the nucleic acid sequence which does not contain the one or more mutations or polymorphisms, or vice versa.

The oligonucleotide probe may comprise a label and binding of the probe may be determined by detecting the presence of the label.

A method may include hybridisation of one or more (e.g. two) oligonucleotide probes or primers to target nucleic acid. Where the nucleic acid is double-stranded DNA, hybridisation will generally be preceded by denaturation to produce single-stranded DNA. The hybridisation may be as part of a PCR procedure, or as part of a probing procedure not involving PCR. An example procedure would be a combination of PCR and low stringency hybridisation.

Binding of a probe to target nucleic acid (e.g. DNA) may be measured using any of a variety of techniques at the disposal of those skilled in the art. For instance, probes may be radioactively, fluoresceritly or enzymatically labelled. Other methods not employing labelling of probe include examination of restriction fragment length polymorphisms, amplification using PCR, RNase cleavage and allele specific oligonucleotide probing. Probing may employ the standard Southern blotting technique. For instance, DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined.

Those skilled in the art are well able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42° C. in 6×SSC and washing in 6×SSC at a series of increasing temperatures from 42° C. to 65° C.

Other suitable conditions and protocols are described in Molecular Cloning: a Laboratory Manual: 3rd edition, Sambrook & Russell (2001) Cold Spring Harbor Laboratory Press NY and Current Protocols in Molecular Biology, Ausubel et al. eds. John Wiley & Sons (1992).

Nucleic acid, which may be genomic DNA, RNA or cDNA, or an amplified region thereof, may be sequenced to identify or determine the presence of polymorphism or mutation therein. A polymorphism or mutation may be identified by comparing the sequence obtained with the database sequence of the component, as set out above. In particular, the presence of one or more polymorphisms or mutations that cause abrogation or loss of function of the polypeptide component, and thus the MMR pathway as a whole, may be determined.

Sequencing may be performed using any one of a range of standard techniques. Sequencing of an amplified product may, for example, involve precipitation with isopropanol, resuspension and sequencing using a TaqFS+Dye terminator sequencing kit. Extension products may be electrophoresed on an ABI 377 DNA sequencer and data analysed using Sequence Navigator software.

A specific amplification reaction such as PCR using one or more pairs of primers may conveniently be employed to amplify the region of interest within the nucleic acid sequence, for example, the portion of the sequence suspected of containing mutations or polymorphisms. The amplified nucleic acid may then be sequenced as above, and/or tested in any other way to determine the presence or absence of a mutation or polymorphism, which reduces or abrogates the expression or activity of the MMR pathway component.

In some embodiments, a cancer may be identified as MMR-deficient by assessing the level of expression or activity of a positive or negative regulator of a component of the MMR pathway, such as MSH2 or MLH1. Expression levels may be determined, for example, by Western blot, ELISA, RT-PCR, nucleic acid hybridisation or karyotypic analysis. In some preferred embodiments, the individual or their tumour may exhibit one or more variations, such as mutations and polymorphisms, in the MSH2 or MLH1 genes.

Mutations and polymorphisms associated with cancer may also be detected at the protein level by detecting the presence of a variant (i.e. a mutant or allelic variant) polypeptide.

### Pharmaceutical Compositions

The active agents disclosed herein for the treatment of MMR-deficient cancer may be administered alone, but it is generally preferable to provide them in pharmaceutical compositions that additionally comprise with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Examples of components of pharmaceutical compositions are provided in Remington's Pharmaceutical Sciences, 20th Edition, 2000, pub. Lippincott, Williams & Wilkins.

Examples of small molecule therapeutics useful for treating MMR-deficient cancers found, by the high-throughput screening reported in the experiments below include:

Methotrexate—(S)-2-(4-(((2,4-diaminopteridin-6-yl) methyl)methylamino)benzamido)pentanedioic acid;
Parthenolide—(1aR,7aS,10aS)-1a,5-dimethyl-8-methylene-2,3,6,7,7a,8,10a,10b-octahydrooxireno[9,10]cyclodeca(1,2-b)furan-9(1aH)-one; and
Menadione—2-methylnaphthalene-1,4-dione.

These compounds or derivatives of them may be used in the present invention for the treatment of MMR-deficient cancer. As used herein “derivatives” of the therapeutic agents includes salts, coordination complexes, esters such as in vivo hydrolysable esters, free acids or bases, hydrates, prodrugs or lipids, coupling partners.

Salts of the compounds of the invention are preferably physiologically well tolerated and non toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, amines, with inorganic acids such as hydrochloric acid, phosphoric acid or sulfuric acid, or organic acids such as acetic acid; citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art.

Derivatives which as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

The term “pharmaceutically acceptable” as used herein includes compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The active agents disclosed herein for the treatment of MMR-deficient cancer according to the present invention are preferably for administration to an individual in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners.

Examples of the techniques and protocols mentioned above can be found in Remington's Pharmaceutical Sciences, 20th Edition, 2000, Lippincott, Williams & Wilkins. A composition may be administered alone or in combination with other treatments, either simultaneously or sequentially, dependent upon the condition to be treated.

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The agents disclosed herein for the treatment of MMR-deficient cancer may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringers Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoule's and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Compositions comprising agents disclosed herein for the treatment of MMR-deficient cancer may be used in the methods described herein in combination with standard chemotherapeutic regimes or in conjunction with radiotherapy. Examples of other chemotherapeutic agents include inhibitors of topoisomerase I and II activity, such as camptothecin, drugs such as irinotecan, topotecan and rubitecan, alkylating agents such as temozolomide and DTIC (dacarbazine), and platinum agents like cisplatin, cisplatin-doxorubicin-cyclophosphamide, carboplatin, and carboplatin-paclitaxel. Other suitable chemotherapeutic agents include doxorubicin-cyclophosphamide, capecitabine, cyclophosphamide-methotrexate-5-fluorouracil, docetaxel, 5-flouracil-epirubicin-cyclophosphamide, paclitaxel, vinorelbine, etoposide, pegylated liposomal doxorubicin and topotecan.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound, and so the actual weight to be used is increased proportionately.

### Experimental Examples

It is often the case that the current approaches to cancer treatment group together similar clinical phenotypes regardless of the differing molecular pathologies that underlie them. A consequence of this molecular heterogeneity is that individuals frequently exhibit vast differences to drug treatments. As such, therapies that target the underlying molecular biology of individual cancers are increasingly becoming an attractive approach (Golub et al., 1999).

On avenue of investigation is to target the loss of tumour suppressor gene function that characterises many cancers. However, loss of tumour suppressor function, in comparison to oncogene activation, presents several problems in the design of potential therapeutic approaches that target these cancers. In the case of oncogene activation, gain of function or activity can potentially be pharmacologically inhibited. Conversely, it is often more technically difficult to efficiently recapitulate tumour suppressor function. However exploiting synthetic lethal interactions with tumour suppressor mutations has been suggested as an attractive approach (Kaelin, 2005, Iorns et al., 2007). Two genes are synthetically lethal if a mutation in either gene alone is compatible with viability but simultaneous mutation of both causes cell death (Kaelin, 2005). As such, the discovery of genes that are synthetically lethal with known cancer-predisposing mutations could aid the identification of novel cancer drug targets. This concept has recently been exemplified by the demonstration that inhibition of the SSB repair enzyme PARP1 is synthetically lethal with BRCA1 or BRCA2 deficiency, with the inhibition of PARP1 profoundly sensitizing BRCA deficient cells. This lethal combination suggests the potential of targeting other DNA repair pathways in the context of other disease-associated mutations (reviewed by Lord et al., 2006).

The DNA mismatch repair (MMR) pathway is integral to the maintenance of genomic stability and is involved in the process of postreplicative repair. MMR corrects DNA polymerase errors such as base-base or insertion/deletion mismatches that form during DNA replication. Unsurprisingly, mutations in MMR genes have often associated with an increase in the frequency of spontaneous mutation and carcinogenesis (Jascur and Boland, 2006, Jiricny, 2006). In particular, germline mutations in the MMR genes MLH1 and MSH2 predispose to hereditary non-polyposis colorectal cancer (HNPCC), which accounts for approximately 5% of all colorectal cancer cases (Jacob and Praz, 2002). To date, 259 MLH1 and 191 MSH2 germline mutations have been associated with an elevated risk of colorectal cancer (Mitchell et al., 2002). Inactivation of the remaining wild-type allele in MLH1 and MSH2 mutant tumours has been shown to occur by somatic mutation (Cunningham et al., 2001, Leach et al., 1993), loss of heterozygosity (LOH; Yuen et al., 2002, Potocnik et al., 2001) or promoter hypermethylation (Cunningham et al., 1998, Potocnik et al., 2001) suggesting that MLH1 and MSH2 act as classical tumour suppressor genes. Significantly, defects in MMR are also observed in 10-25% of sporadic cancers, often as a result of aberrant MLH1 promoter methylation (Arnold et al., 2003, Bettstetter et al., 2007, Peltomaki, 2003).

Given that the application of synthetic lethal interactions of DNA repair proteins is showing therapeutic promise, we aimed to determine whether synthetic lethal interactions could be applied to loss of MMR function and whether such an approach could be exploited therapeutically.

Studies in yeast suggest a synergistic hypermutability and lethality caused by a combination of proofreading polymerase mutations and MMR defects (Morrison et al., 1993, Tran et al., 1999, Argueso et al., 2002, Pavlov et al., 2001). However the relationship between DNA polymerases and MMR in mammals is less clear. Studies in mice indicate that Msh2 deficiency results in the accumulation of oxidative base damage (DeWeese et al., 1998, Colussi et al., 2002) as does deficiency in the DNA polymerase β (POLβ), a component of the base excision repair (BER) pathway (Yoshimura et al., 2006, Horton et al., 2002).

Given the suggestion of synthetic lethality in model organisms, we examined whether the MMR gene MSH2 and POLP were synthetically lethal in human cells.

### Experimental Procedures

### Cell Lines

The human endometrial cell lines HEC59 and HEC59+chr2 were employed. Hec59+Chr2 and Hec59 cells were grown in DMEM F12 (1:1) supplemented with FCS (10%, v/v), glutamine and antibiotics. The human colon cancer cell line HCT116 and HCT116+chr3 were grown in McCoys 5A supplemented with FCS (10% v/v), glutamine, and antibiotics. Cells containing human chromosome 2 were cultured under selective pressure of 400 μg/mL geneticin (G418 sulfate, Life Technologies, UK). Hela cells were grown in DMEM, supplemented with FCS (10%, v/v), glutamine and antibiotics. The human ovarian tumor cell lines A2780 cp70+chr3/A2 and A2780 cp70+chr3/E1 were maintained in RPMI 1640 supplemented with FCS (10%, v/v), glutamine, and antibiotics. Cells were cultured under selective pressure of 200 μg/mL Hygromycin B (Invitrogen, UK). HeLa cells were grown in DMEM, supplemented with FCS (10% v/v), glutamine and antibiotics. snRNA expressing cells were established by infecting HeLa cells with shRNA expressing empty or hMSH2 vectors, which were generated by PCR amplification of 97mer DNA oligonucleotides as described (Paddison et al., 2004) and cloned into the LMP vector (Dickins et al., 2005) by EcoRI/XhoI sucloning. shRNA sequences were as follows:

shMsh2
TGCTGTTGACAGTGAGCGCCTCAGTGAATTAAGAGAAATATAGTGAAGCC
ACAGATGTATATTTCTCTTAATTCACTGAGATGCCTACTGCCTCGGA

### Protein Analysis

Cell pellets were lysed in 20 mmol/L Tris (pH 8), 200 mmol/L NaCl, 1 mmol/L EDTA, 0.5% (v/v) NP40, 10% (v/v) glycerol, and protease inhibitors. Immunoprecipitations were performed by incubating Protein G beads (Sigma), 1-2 mg of precleared cell lysate and anti-POLB antibody (ab3181, Abcam; dilution 1:100) overnight at 4° C. Beads were subsequently washed three times in cold lysis buffer, after which 2× loading buffer was added and the samples were boiled for 5 min before SDS-PAGE. For western blotting, lysates were electrophoresed on Novex precast gels (Invitrogen) and immunoblotted using the following antibodies: anti-MSH2 (Ab-1, Calbiochem), anti-POLB (ab3181, Abcam), anti-MLH1 (ab9144, Abcam), anti-POLG (Novus), anti-PCNA (SC7907, Santa-Cruz), anti-Cytochrome C (Pharmagen) anti-OGG1 (NB100-106, Novus biologicals), anti-CHIP (ab39559, Abcam) and anti-β-tubulin, (T4026, Sigma). This was followed by incubation with anti-IgG-horseradish peroxidase and chemiluminescent detection (SuperSignal West Pico Chemiluminescent Substrate, Pierce). Immunoblotting for (1-tubulin was used as a loading control.

### In Vitro OGG1 Assay

OGG1 glycosylase activity was using the OGG1 assay kit (Sigma Aldrich, UK). Briefly, protein was isolated from transfected cells as indicated in FIG. 4B. The substrate is a 23 oligonucleotide containing 8-OHdG at its 11th base, labeled with ³²P at its 5′ end, and annealed to its complementary strand (containing dC at the opposite base position to the 8-OHdG). Upon cleavage of the substrate by the OGG1 enzyme, the oligonucleotides were electrophoresed on a 15% polyacrylamide denaturing (7 M UREA) PAGE gel, followed by autoradiography.

Detection of oxidative DNA lesions by Immunofluorescence Transfected cells were seeded onto glass slides. 48 hr post transfection, cells were fixed for 15 min with 4% paraformaldehyde in PBS. Slides were then permeabilized with TBS/Tween-20 and followed by serial washes in methanol solutions, prior to washing with TBS/Tween-20, blocking for 1 h at 37° C. and then incubated with FITC labeled 8-OHdG binding protein, for 24 h at 4° C. (BiotrinOxyDNA Test, Biotrin, UK). Cover slips were stained with DAPI, mounted and viewed using a Leica TCS-SP2 confocal microscope:

### Use of RNA Interference to Assess Synthetic Lethality

Cells were transfected with short interfering RNA (siRNA) (Qiagen, UK) targeting the following genes (target sequences shown):

| | |
|---|---|
| POLγ*1, | 5′-CACGAGCAAATCTTCGGGCAA-3′; |
| POLγ*2, | 5′-CAGATGCGGGTCACACCTAAA-3′; |
| POL β*1, | 5′-CAAGATATTGTACTAAATGAA-3′; |
| POL β*2, | 5′-TACGAGTTCATCCATCAATTT-3′; |
| POLι*1, | 5′-ACCGGGAACATCAGGCTTTAA-3′; |
| POLι*2, | 5′-GCGGTTTATTAAGCTCTTCTA-3′; |
| POLη*1, | 5′-ATCCATTTAGGTGCTGAGTTA-3′; |
| POLη*2, | 5′-CTGGTTGTGAGCATTCGTGTA-3′; |
| POLε*1, | 5′-CCGCATCATCCTCTGTACAAA-3′; |
| POLε*2, | 5′-CCGCCTCTCCATTGACCTGAA-3′; |
| OGG1*1, | 5′-CGGGACCTACACCTCAGGAAA-3′; |
| OGG1*2, | 5′-CACCGTGTGGGCGAGGCCTTA-3′; |
| CHIP*1, | 5′-CCGCGGAGCGUAGAGAGGGA-3′; |
| CHIP*2, | 5′GCAUUGAGGCCAAGCACGA-3′; |
| DHFR*1, | 5′-TACGGAGAAACTGAACTGAGA-3′; |
| DHFR*2, | 5′-AACCTCCACAAGGAGCTCATT-3′; |
| SCYL *1, | 5′-CCCGTTGGGAATATACCTCAA-3′; |
| SCYL1 *2, | 5′-CAACCGCTTTGTAGAAACCAA-3′; |
| MSH2*1, | 5′-CCCATGGGCTATCAACTTAAT-3′; |
| MSH2*2, | 5′-TCCAGGCATGCTTGTGTTGAA-3′; |
| siControl, | 5′-CATGCCTGATCCGCTAGTC-3′. |

For 96-well plate-based cell viability assays, HeLa, HCT116, HCT116+chr3, Hec59 and Hec59+chr2 cells were transfected with individual siRNA using Lipofectamine 2000 (Invitrogen, UK) according to manufacturer's instructions. A2780 cp70+chr3/A2 and A2780 cp70+chr3/E1 cells were transfected with individual siRNA using Lipofectamine RNAi Max (Invitrogen) according to manufacturer's instructions. As a control for each experiment, cells were left un-transfected or transfected with a non-targeting Control siRNA and concurrently analysed. Twenty-four hours after transfection, cells were plated into replica plates. Cell viability was measured five days after transfection using the 96-well plate CellTiter-Glo Luminescent Cell Viability Assay kit (Promega, UK) according to the manufacturer's instructions. Survival fractions were calculated by dividing the cell viability for a given transfection by the cell viability of the siControl siRNA-transfected cells. All transfections were carried out in triplicate.

For clonogenic assays, exponentially growing cells were seeded at various densities in six-well plates. Cells were transfected with siRNA as before. Cell medium was replaced every four days. After ten to fourteen days, cells were fixed in methanol, stained with crystal violet, and counted. The plating efficiencies were calculated as the number of colonies divided by the number of cells plated for each siRNA transfection. The surviving fraction (SF) for a given sample was calculated as the plating efficiencies for each siRNA transfection divided by the plating efficiencies of siControl siRNA transfected cells. All transfections were carried out in triplicate.

Validation of Gene Silencing by siRNA

Transfected cell pellets were lysed in 20 mmol/L Tris (pH 8), 200 mmol/L NaCl, 1 mmol/LEDTA, 0.5% (v/v) NP40, 10% (v/v) glycerol, and protease inhibitors. Lysates were electrophoresed on Novex precast gels (Invitrogen, UK) and immunoblotted using the following antibodies: anti-MSH2 (Ab-1, Calbiochem), anti-POL β (ab3181, Abcam), anti-DHFR (ab49881, Abcam), anti-SCYL1 (Abgent) and anti-β-tubulin, (T4026, Sigma). This was followed by incubation with anti-IgG-horseradish peroxidase and chemiluminescent detection (enhanced chemiluminescence, Amersham, UK). Immunoblotting for β-tubulin was used as a loading control.

### Quantitative RT-PCR

Quantification of RNA expression was measured by real time RT-PCR. Total RNA was extracted from cells with Trizol (Invitrogen) according to manufacturer's instructions. Total RNA from patient biopsies was purified from 10 μm sections using the High Pure RNA Paraffin Kit (Roche Diagnostic Ltd). cDNA was synthesized using Omniscript Reverse Transcriptase System for RT-PCR (Qiagen) with oligo dT as per manufacturer's instructions. Assay-on-Demand primer/probe sets were purchased from Applied Biosystems (Foster City, Calif.). Real-Time qPCR was performed on the 790DHT Fast Real-Time PCR System (Applied Biosystems), with endogenous control β-Actin. Standard curves were calculated for all reactions with serial dilutions of control Hec59+chr2 cells to calculate reaction efficiency. Gene expression was calculated relative to expression of β-Actin endogenous control, and adjusted relative to expression in control Hec59+chr2 cells. Samples were quantified in triplicate.

### Measurement of 8-OHdG

Genomic DNA was extracted using the Qiamp DNA isolation kit (Qiagen) and digested with nuclease P1. Mitochondrial and nuclear DNA was extracted using the mitochondrial DNA isolation kit (ab65321, Abcam). A commercially available ELISA kit from Cell Biolabs was used to determine levels of 8-OHdG in isolated DNA. The assays were performed according to the manufacturer's instructions. The 8-OHdG standard (0.078-20 ng/ml) or 10 μg DNA from siRNA transfected cells was incubated with a 8-OHdG monoclonal antibody in a microtiter plate precoated with 8-OHdG.

Addition of 3,35,5-tetramethylbenzidine to replicate samples was followed by measurement of absorbance at 450 nm. Standard curves were calculated for all reactions with serial dilutions of 8-OHdG standard to calculate reaction efficiency. Samples were assayed in triplicate.

### Immunohistochemical Staining

Four μm sections were cut from the formalin fixed paraffin embedded samples for the purpose of immunohistochemistry. Immunohistochemistry was performed for anti-MSH2 antibody (Zymed; clone FE11, dilution 1/400; Antigen retrieval: ER120 minutes) and anti-MIiH1 antibody (BD Transduction Laboratories; clone G168-15, dilution 1/150; Antigen retrieval: ER240 minutes) on an automated platform (BondMax™ system-Vision BioSysteme™). Staining was performed according to the protocol as listed above with the antibody details. A polymer detection system was selected to avoid non-specific endogenous biotin staining. A section of normal colon tissue was used as a positive control, and negative controls were performed by replacing the antibody with Tris buffered saline. Cases with unequivocal nuclear staining were considered positive. Validation of staining was confirmed by expression in normal colonic mucosa cells, normal epithelial cells, stromal cells or lymphocytes (Mackay et al., 2000).

### Compound Inhibitor Screen

Compound libraries were purchased from Prestwick Chemicals (Saffron Walden, Essex, UK). Validation experiments were carried out with Methotrexate (Biomol International L.P.) and Menadione and Partenolide (Prestwick chemicals). Cells were plated in 96-well plates. After 12 hr incubation, cells were exposed to compound or equimolar DMSO and re-treated every 48 hrs. Cell viability was measured six days later using the CellTitre Glo assay (Promega) according to the manufacturer's instructions. Validation of hit compounds was performed by clonogenic assays. Exponentially growing cells were seeded at various densities in six-well plates. Cells were treated with increasing concentrations of the compound. Cell medium was replaced every four days. After ten to fourteen days, cells were fixed in methanol, stained with crystal violet, and counted. The plating efficiencies were calculated as the number of colonies divided by the number of cells plated for each compound treatment. The surviving fraction (SF) for a given sample was calculated as the plating efficiencies for each compound treated cells divided by the plating efficiencies of DMSO treated cells.

### Results

hMSH2 is Synthetically Lethal with POLβ

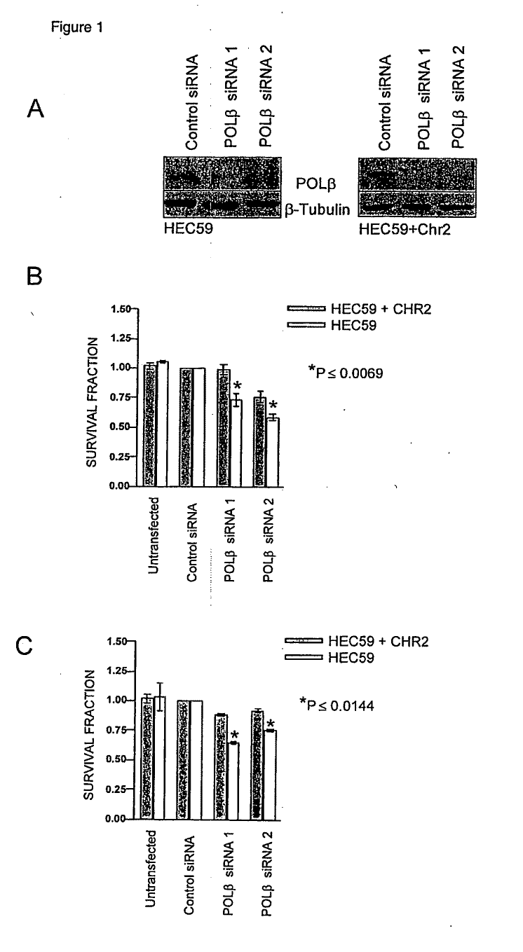

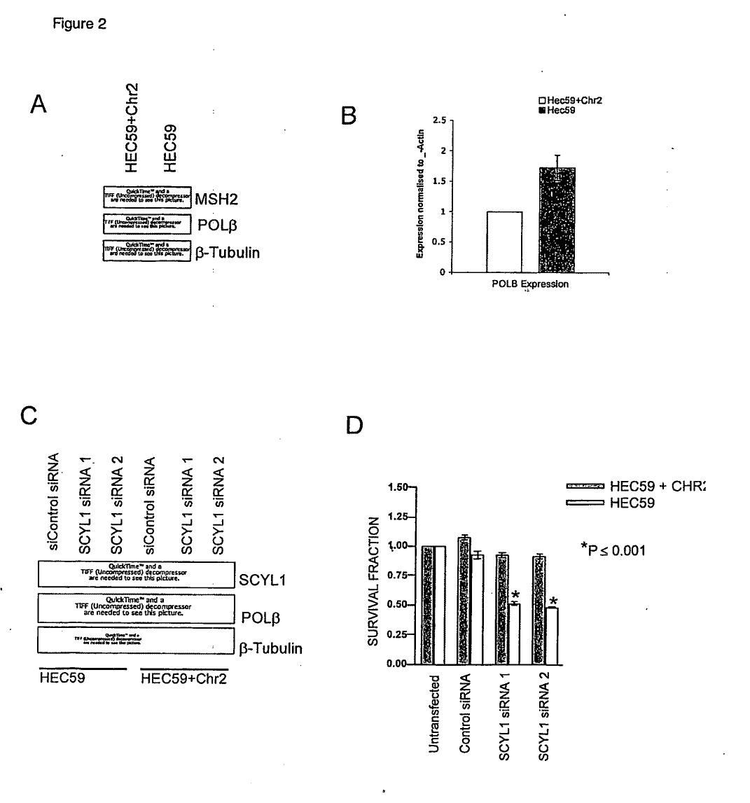

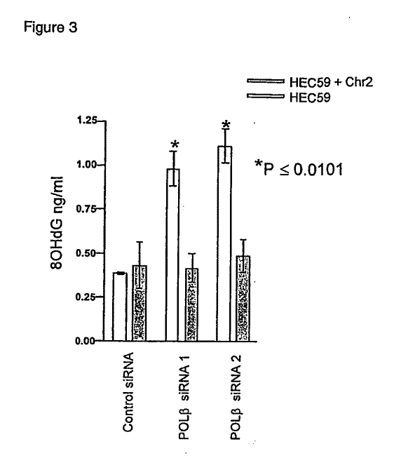

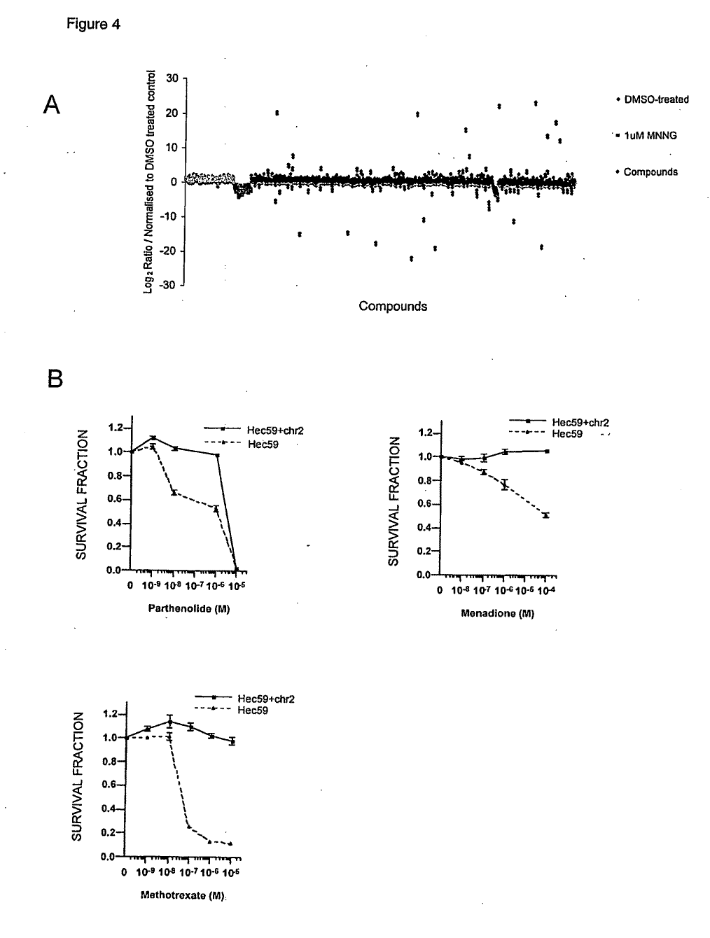

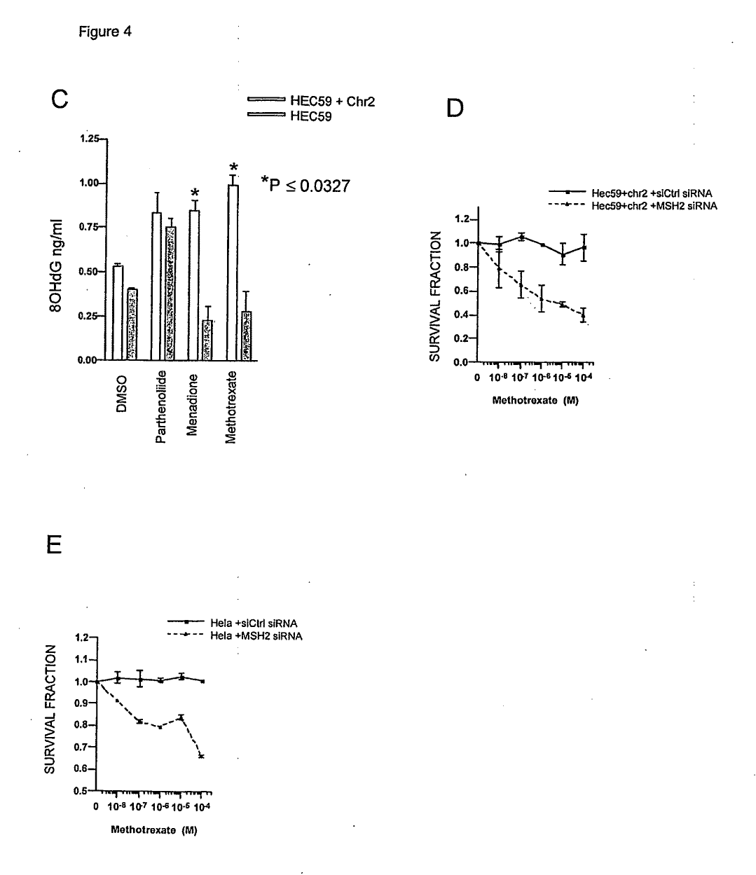

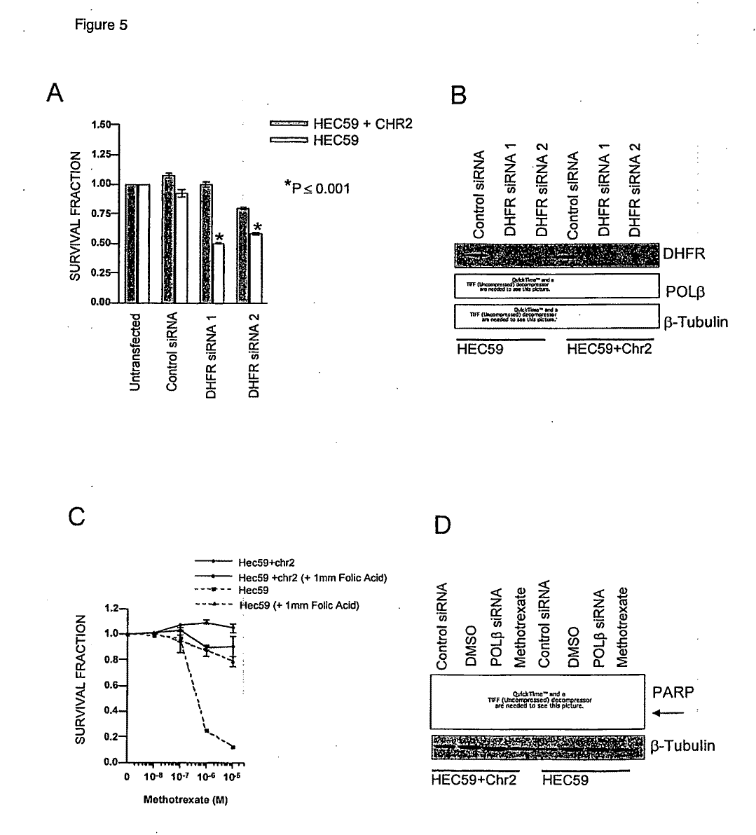

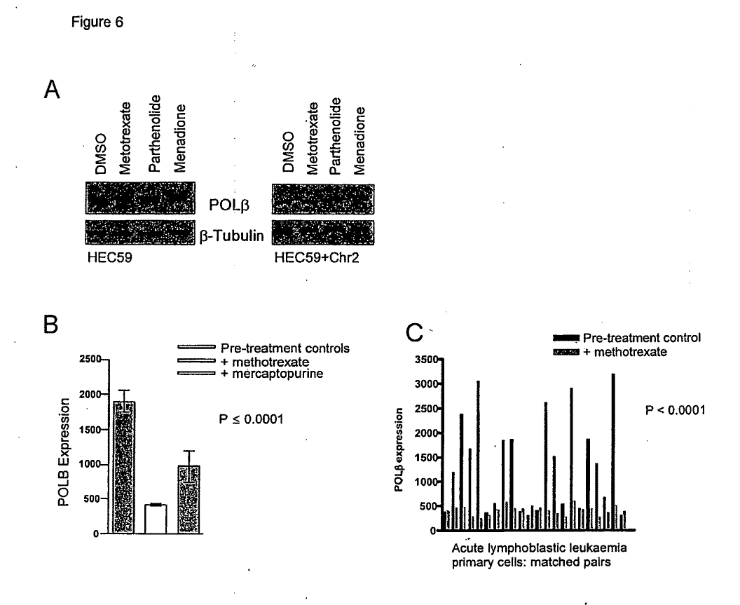

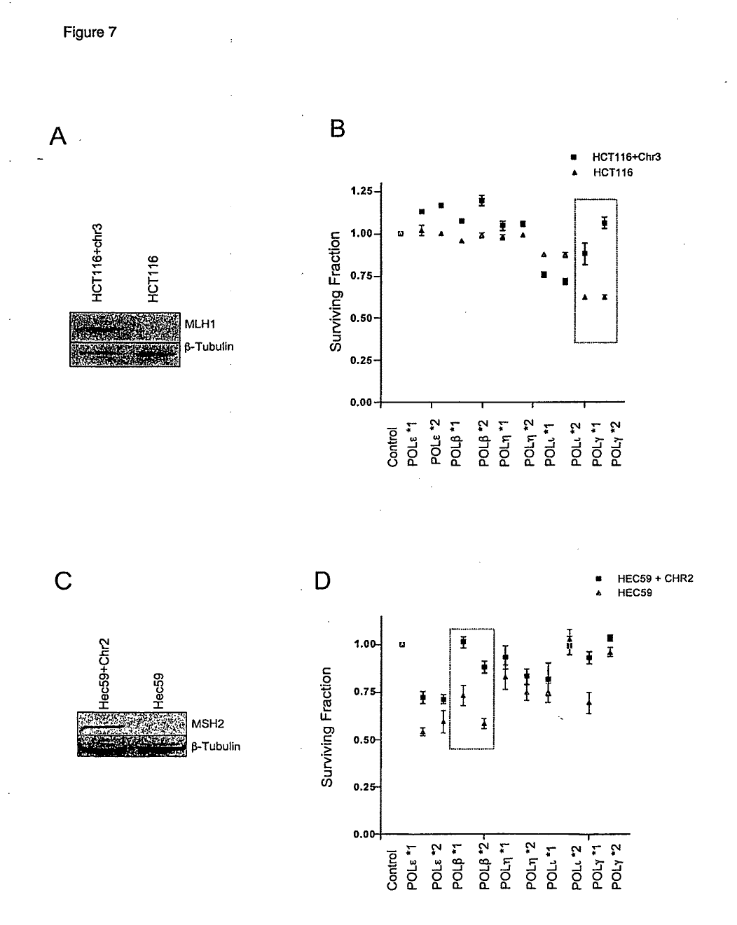

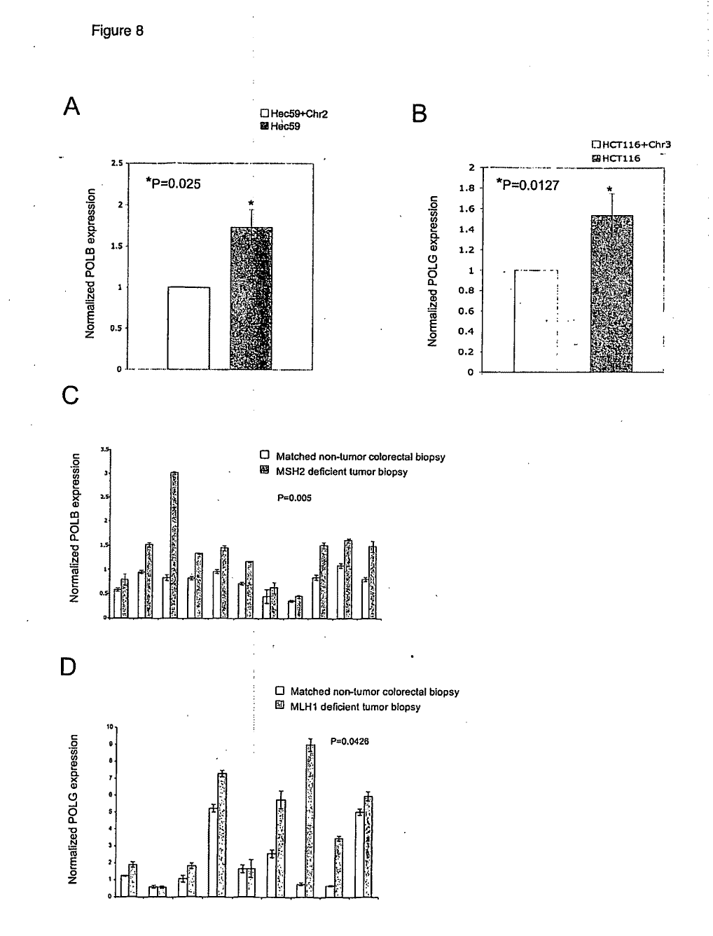

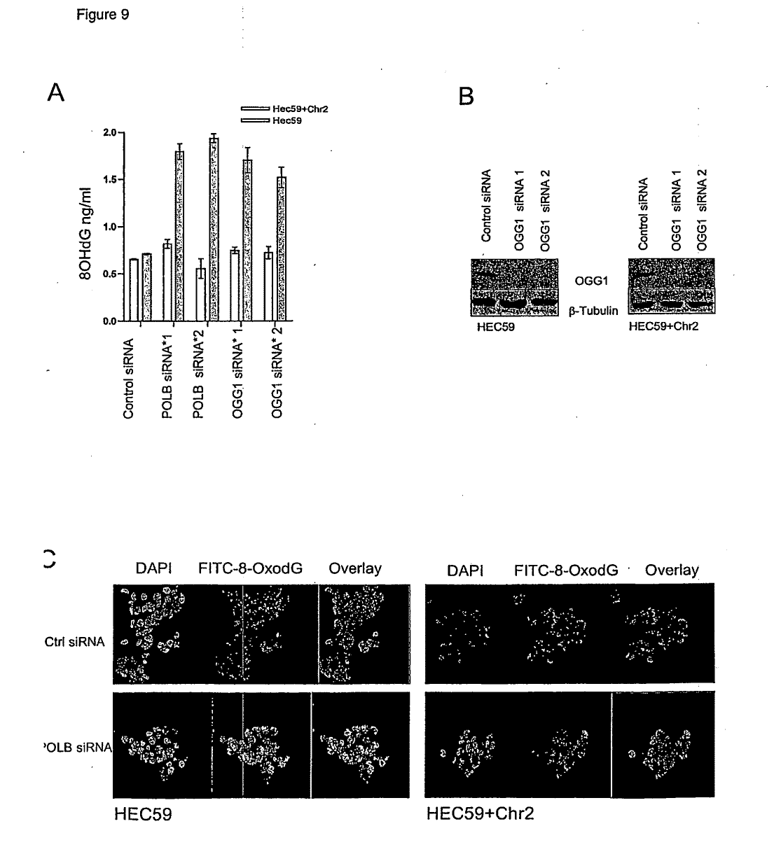

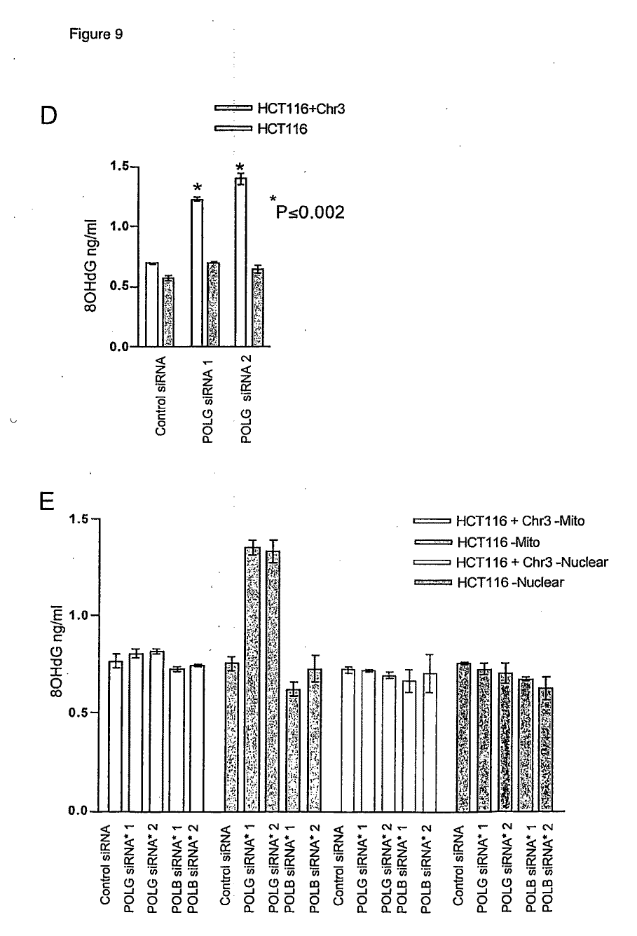

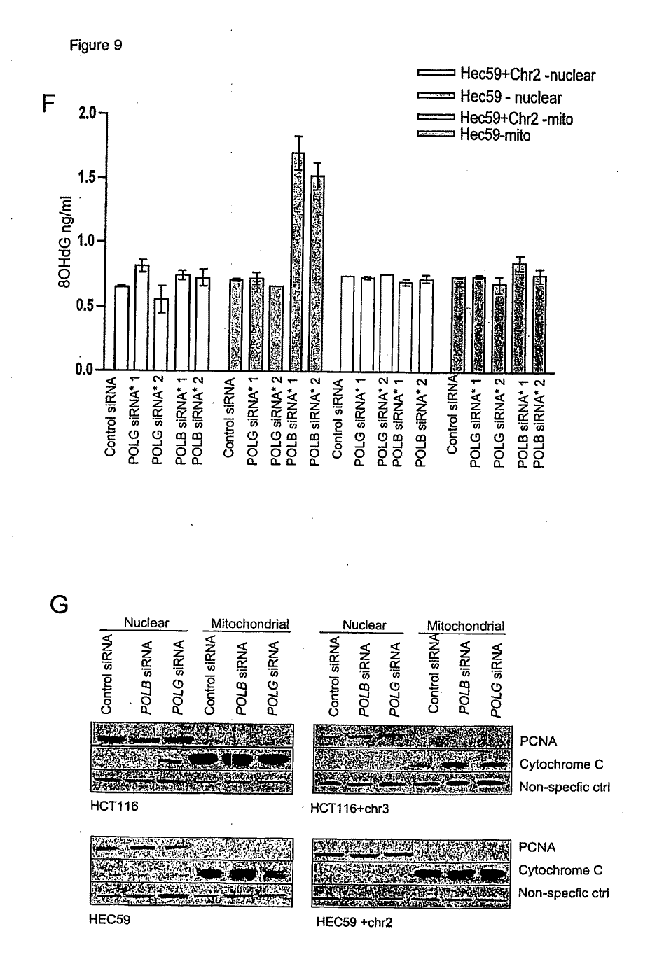

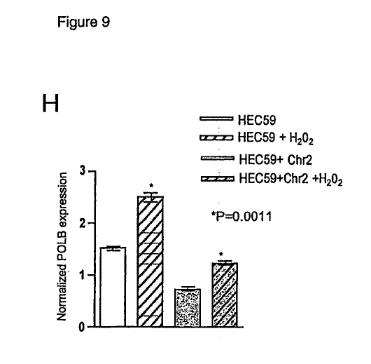

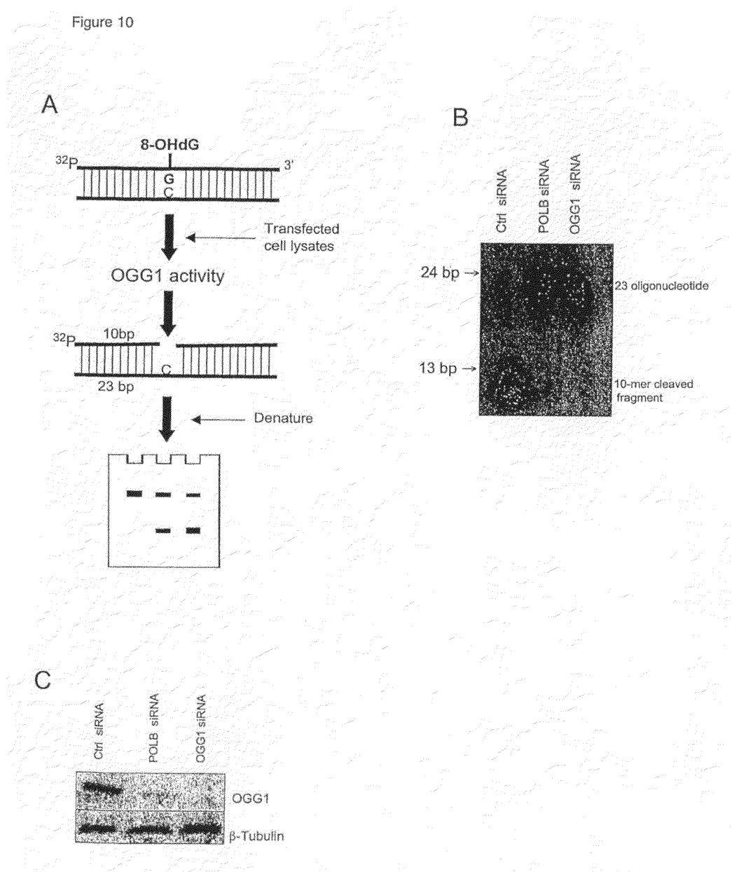

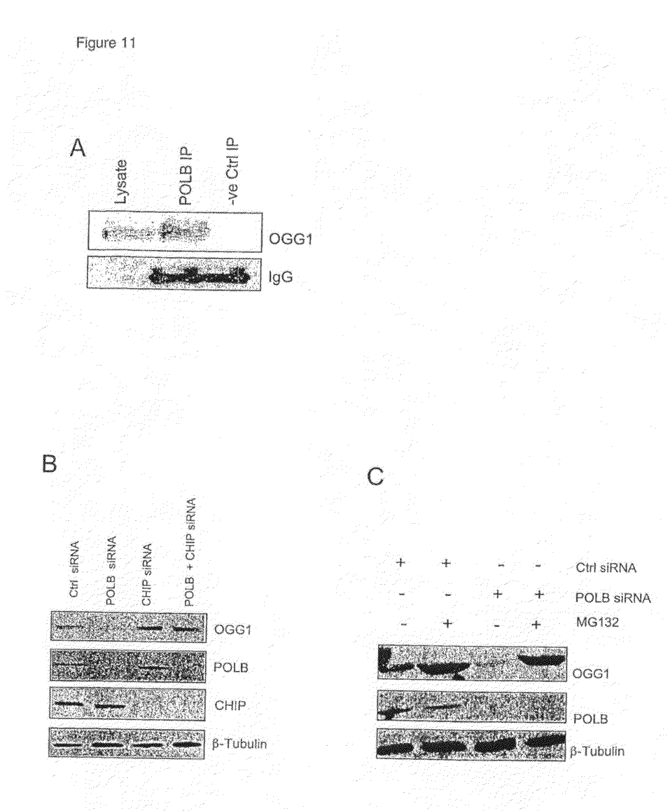

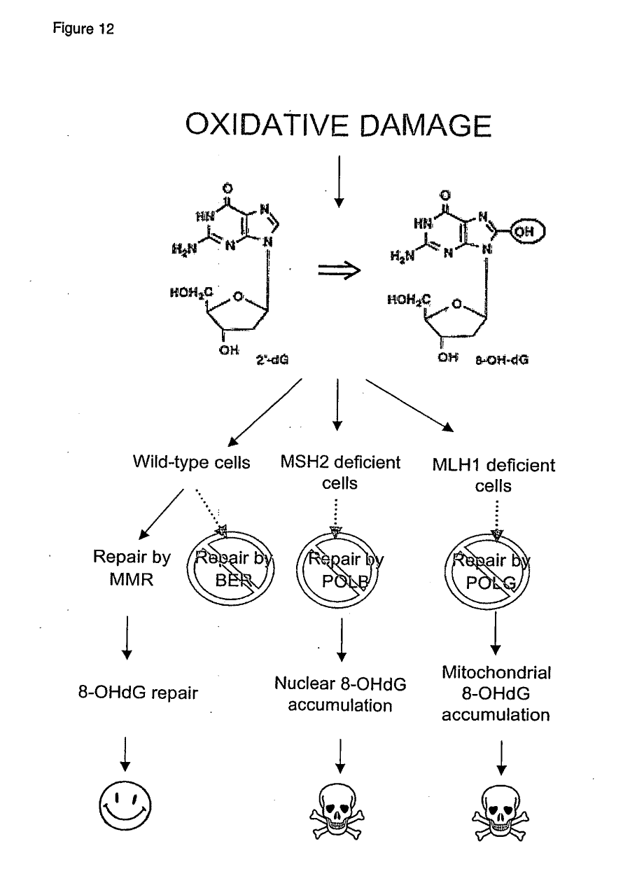

Based on results obtained in yeast and the observation that Msh2 deficiency in mice results in increased oxidative damage accumulation, the possibility of a synthetic lethal interaction between the MMR gene MSH2 and the oxidative damage associated polymerase β in humans was examined. These experiments used the previously characterised human endometrial cell line, Hec59, which bears inactivating mutations in both alleles of MSH2 (Umar et al., 1997). In order to clearly identify synthetic lethal interactions, the comparison of isogenic cell lines is essential (Kaelin, 2005). Accordingly, the Hec59 cell line was compared to an isogenic cell line in which wild type MSH2 was introduced by the transfer of human chromosome 2 (Hec59+Chr2). Both cell lines were transfected with short interfering (si)RNA directed against POLβ. To eliminate the possibility of off-target false positive results, two siRNA species targeting different sequences within the POLβ transcript were used. As a control for each experiment, cells were left un-transfected or transfected with a non-targeting Control siRNA (siControl) and concurrently analysed. Western blot analysis confirmed reduction in POLβ protein expression after siRNA transfection (FIG. 1A). Cell viability was assessed after six days by ATP assay. The surviving fraction of the MSH2 deficient Hec59 cells transfected with POLβ siRNA*1 and POLβ siRNA*2 resulted in a statistically significant reduction (P≦0.0069) in viability as compared to the similarly transfected MSH2 proficient Hec59+chr2 cells (FIG. 1B), suggesting a synthetic lethal synergy between downregulation of POLβ and MSH2 deficiency. To eliminate the possibility of assay-specific effects, this synthetic lethal relationship was also validated by clonogenic assay, which is believed to be the gold standard assay to measure cell death and permanent growth inhibition for cells in vitro (FIG. 1C).

MSH2 Deficiency is Associated with Increased POLβ Expression

Having observed that loss of MSH2 and POLβ are synthetically lethal, the mechanism associated with this synergy was investigated further. The expression level of POLβ in MSH2 proficient and deficient cell lines was examined by Western blot analysis and qRT-PCR (FIGS. 2A & B). It was observed that POLβexpression is upregulated in the MSH2 deficient cell line Hec59 at both the protein and transcript levels, in comparison to the MSH2 proficient Hec59+chr2 cell line. This suggested that in the absence of functional MSH2, POLβ is upregulated and may compensate for MMR deficiency. Previous work has demonstrated that ectopic expression of the telomerase transcriptional element-interacting factor, TEIF (also known as SCYL1) in HELA cells can upregulate both levels of endogenous POLβ mRNA and protein and consequently may increase resistance to the oxidative stress of H₂O₂ (Zhao et al., 2005). Therefore, the mechanism by which POLβ expression is regulated in Hec59 cells was explored, using two siRNA targeting SCYL1 in transient transfection experiments (FIG. 2C). Inhibition of SCYL1 expression resulted in the reduction of POLβ expression, suggesting that SCYL1 is likely responsible for the upregulation of POLβ in the absence of MSH2. Consequently, experiment to test whether SCYL1 inhibition would result in loss of POLβ expression, leading to lethality in combination with MSH2 deficiency, were carried out. To this end, MSH2 deficient and proficient Hec59 and Hec59+chr2 cells were transfected with two SCYL1 siRNA and cellular viability was analysed after six days. Suppression of SCYL1 expression by siRNA did indeed result in a synthetic lethal synergy with MSH2 deficiency in Hec59 cells (FIG. 2D), further supporting the hypothesis that increased expression of POLβ in the absence of functional MSH2 is regulated by SCYL1 and this regulation is necessary for cellular viability in the absence of MSH2.

Increased 8-OHdG Accumulation Correlates with POLβ and MSH2 Deficiency

Both MSH2 and POLβ have previously been implicated in the prevention of 8-OHdG accumulation and oxidative DNA damage repair. Therefore, experiments were carried out to examine whether the MSH2/POLβ synthetic lethal synergy was due to an increased accumulation of oxidised DNA lesions. 8-OHdG levels were quantified in POLβ silenced cells using a highly sensitive 8-OHdG competitive ELISA assay (Cell Biolabs). This assay system uses a monoclonal antibody targeting 8-OhdG to quantify this oxidised base. A significant increase in accumulation of the 8-OHdG oxidation lesion in MSH2 deficient Hec59 cells upon POLβsilencing was observed (FIG. 3). No increase was observed in the isogenically matched MSH2 proficient cell line Hec59+chr2 after POLβ inhibition. This observation strongly suggests that increased accumulation of 8-OHdG is a likely mechanism for the synthetic lethal relationship between MSH2 and POLβ. Genetic alterations within the MSH2 deficient cells, increase their requirement for POLβ to repair oxidised lesions occurring in the cell, relative to MSH2 proficient cells. Therefore, inhibition of both MSH2 and POLβ results in an accumulation of un-repaired oxidized lesions, which is increasingly toxic to the cell. This creates an opportunity for selectivity based on loss of a repair mechanism for 8-OHdG lesions in MSH2 mutant cells upon POLO inhibition, while sparing MSH2 proficient cell lines.

Small Molecules Inducing Oxidative Damage Cause Synthetic Lethality with MSH2 Deficiency

Having identified a MSH2/POLβ synthetic lethal relationship and a strong correlation between this lethality and 8-OHdG accumulation, this information was utilised to identify existing clinical agents that may be of efficacy in treatment of MMR deficient cancer. To this end, the isogenically matched MSH2 deficient and proficient cell lines Hec59 and Hec59+chr2 were screened with a compound library consisting of 1120 small molecules, 90% of which are marketed drugs and 10% of which are bioactive alkaloids (FIG. 4A). This high-throughput screen identified three compounds, Parthenolide, Menadione and Methotrexate, which show selectivity for MSH2 deficient cells and have previously been reported to induce oxidative damage (Mardi et al., 2007, Cojocel et al., 2006 and Rajamani et al., 2006). Validation of the screen results was performed using clonogenic assays to eliminate assay-specific effects and to establish clear dose-response relationships (FIG. 4B). In addition, cells were treated with each of the three hit compounds for three days after which isolated DNA was analysed for 8-OHdG accumulation using an ELISA assay (FIG. 4C). In Hec59+chr2 cells (MSH2 proficient), 8-OHdG accumulation did not increase after treatment with menadione and methotrexate suggesting that these cells have the ability to repair damage induced by oxidative damage. In contrast, in the MSH2 deficient Hec59 cell line, the amount of 8-OHdG increased significantly after drug treatment indicating that MSH2 is required for efficient repair of these oxidative lesions. Parthenolide, is not a potential selective therapeutic agent for MSH2 deficiency, due to high toxicity as a result of increased 8-OhdG accumulation in both cell lines. To ensure that the observed MSH2 deficiency and oxidative damage lethal synergy is due to MSH2 rather than the specific cell lines used in the study, the MSH2 proficient Hec59+chr2 cells and also the human cervical cancer HeLa cell line were transfected with either siCtrl siRNA or MSH2 siRNA and subsequently treated with increasing concentrations of methotrexate. FIGS. 4C & D illustrate that transfection of Hec59+chr2 cells or HeLa cells with MSH2 siRNA is similarly synthetically lethal with methotrexate as observed with the Hec59 MSH2 deficient cell line.

Methotrexate Treatment Inhibits POLβ Expression through Inhibition of Folate Synthesis.

Methotrexate inhibits dihydrofolate reductase (DHFR), an enzyme that is part of the folate synthesis metabolic pathway (Goodsell, 1999). To establish whether synthetic lethality with MSH2 deficiency is due to inhibition of folate production or a non-specific action of the compound, Hec59 and Hec59+chr2 cells were transfected with siRNA targeting DHFR. FIG. 5A illustrates that MSH2 deficiency is synthetically lethal with DHFR, strongly suggesting that the effect of methotrexate is due to the specific action of folate synthesis inhibition by the compound.

To validate this observation, MSH2 deficient and proficient Hec59 and Hec59+chr2 cells were treated with methotrexate in addition to folic acid (FIG. 5C). Addition of folic acid in concert with methotrexate, in large part rescues the lethal phenotype observed in the Hec59 MSH2 deficient cells, supporting our model for the mechanism of action by methotrexate.

To further examine the mechanism of synthetic lethality of methotrexate with MSH2 deficiency, cells treated with methotrexate were immunoblotted for POLβ expression. Interestingly, methotrexate treatment resulted in a reduction of POLβ expression in Hec59 and Hec59+chr2 cells (FIG. 5D). This observation further validates the synthetic lethality observed with MSH2 and POLIS deficiency. This leads to the conclusion that silencing of POLIS through methotrexate treatment or by siRNA transfection results in a synthetic lethal synergy with MSH2 deficiency, due to an increased accumulation of 8-OHdG oxidative lesions, as illustrated in FIG. 6.

MMR Deficiency is Synthetically Lethal with Silencing of DNA Polymerases

It has been previously suggested that replication errors are first acted upon by DNA proofreading polymerases and those that remain become substrates for MMR. This model is further supported by the observation that, in yeast, mutations in the proofreading polymerase pol3-01 (the orthologue of human POLD catalytic subunit) are synthetically lethal with loss of the orthologues of the MMR genes EXOI, MSH6, MSH2, MLH1 and PMS1 (Morrison et al., 1993; Tran et al., 1999; Argueso et al., 2002; Tran et al., 1997). Similarly the yeast Msh6-PolH double mutant is not viable (Pavlov et al., 2001). Msh2-pol2-4 (PolE) mutants are viable but have mutation rates 50-fold higher than those of either single mutant individually (Tran et al., 1997). Here we investigated whether synthetic lethal interactions between MMR and DNA polymerases are conserved in higher eukaryotes, as this might indicate that these enzymes are potential therapeutic targets in cancers with MMR defects.

To assess synthetic lethal interactions, we used isogenic models of MLH1 or MSH2 deficiency. To model MLH1 deficiency, we used the previously characterised human colon adenocarcinoma cancer cell line HCT116 cell line, which has a homozygous mutation of the MLH1 gene. As a comparator, we used the MLH1 proficient HCT116+chr3 cell line; transfer of human chromosome 3 into HCT116 cells results in the expression of functional MLH1, complementing the MMR defect (FIG. 7A). To model MSH2 deficiency, we used the previously characterised human endometrial cell line HEC59, which harbours two different loss-of-function MSH2 nonsense mutations. As a comparator, we used the MSH2 proficient HEC59+chr2 cell line; transfer of human chromosome 2 into this cell line results in the expression of functional MSH2 (Umar et al., 1997) (FIG. 7C). All cell lines were screened with a panel of short interfering (si)RNA targeting a number of DNA polymerases including POL ε, β, η, t and γ. To minimise the possibility of identifying off-target effects, two siRNA species with differing target sequences were used for each polymerase. As a control for each experiment, cells were-transfected with a nontargeting Control siRNA and concurrently analysed. Cell viability was assessed five days after siRNA transfection. This small-scale siRNA screen identified a number of synthetic lethal interactions, as represented by significantly more death in MLH1 or MSH2 deficient cells compared to their MMR proficient comparators (FIGS. 7B & D).

Most strikingly, silencing of POLB in MSH2 deficient cells and silencing of POLG in MLH1 deficient cells resulted in a significant decrease in viability, when compared to their MMR proficient counterparts. Both of these synthetic lethal interactions were validated in additional isogenic models.

MMR Deficiency is Associated with Increased DNA Polymerase Expression

Synthetic lethal interactions between pathways may be as a consequence of functional compensation and are often associated with increased expression of the compensatory pathway. To further investigate the MSH2-POLB and MLH1-POLG interactions we measured POLB and POLG mRNA levels in cells with either MSH2 or MLH1 deficiencies. POLB mRNA levels were significantly higher (P=0.025) in the MSH2 deficient Hec59 cell line, compared to the MSH2 proficient Hec59+chr2 cell line. Similarly, POLG mRNA expression levels in the MLH1 deficient HCT116 cells were significantly higher (P=0.0127), in comparison to the HCT116+Chr3 cells (FIG. 8B). Upregulation of polymerase expression were validated in additional isogenic models (Suppl. FIGS. 8A & B). Taken together with the synthetic lethal interactions, this expression data suggested that in the absence of functional MMR, levels of specific polymerases are elevated and this may compensate in some way for MMR deficiency.

To further validate these observations, we assessed POLB and POLG mRNA levels in colorectal tumours from patients with MMR gene deficiency. In tumors with mutations in MLH1 or MSH2, inactivation of the remaining wild-type allele has been shown to occur by somatic mutation, loss of heterozygosity (LOH), or promoter hypermethylation. We measured POLB mRNA levels in MSH2 deficient colorectal tumor biopsies. POLB transcript levels in tumor biopsies were compared to those in matched MSH2 positive normal colon biopsies from the same individuals. This analysis indicated that POLB mRNA expression levels were significantly higher (P=0.005) in MSH2 deficient tumors than in the corresponding MSH2-expressing non-tumour biopsies (FIG. 8C).

Similarly, we analysed POLG expression in matched tumour and normal colon biopsies from nine patients with MLH1 deficient tumours, and found that POLG was significantly upregulated (P=0.042) in MLH1 deficient tumours, compared to normal tissue (FIG. 8D). This suggested that upregulation of specific DNA polymerases in the context of MMR deficiency was unlikely to be a cell culture-specific observation. Taken together, these results indicate that in the absence of MMR, specific DNA polymerases are upregulated and may possibly compensate for particular MMR gene deficiencies in cancer cells and colorectal tumours.

Increased 8-OHdG Accumulation Correlates with Selective Lethality with MMR Deficiency

A number of studies suggest a role for MSH2 and POLB in the prevention of 8-OHdG accumulation. Analysis of 8-OHdG:C repair in tissues from Polb+/− mice indicated that there is a significant reduction in the ability to repair this form of DNA damage. Moreover, Polb+/− mice were more sensitive than wild-type mice to oxidative stress induced by 2-Nitropropane. Significantly, Polb null mouse fibroblasts demonstrate hypersensitivity to hydrogen peroxide and other reactive oxygen species-generating agents over time in culture. Therefore, we investigated whether the MSH2/POLB synthetic lethality might be explained by the rapid accumulation of 8-OHdG lesions beyond a threshold incompatible with viability. We tested this hypothesis by determining the levels of 8-OHdG accumulation by ELISA. POLB silencing caused a significant increase in 8-OHdG levels in MSH2 deficient Hec59 cells, compared to that in MSH2 proficient cells (FIG. 9A). A similar increase was observed upon siRNA silencing of OGG1, the glycosylase required for the cleavage of 8-OHdG lesions (FIG. 9A). No significant increase was observed in the similarly transfected MSH2 proficient cell line, Hec59+chr2. These observations were further supported by immunofluorescence detection of 8-OHdG using a fluorescein-tagged 8-OHdG-binding protein (FIG. 9C). Increased immunofluoresence of the fluorescein tagged 8-OHdG-binding protein was observed upon POLB silencing in MSH2 deficient cells, as compared to the similarly transfected MSH2 proficient cell line. Taken together, these results suggest that increased accumulation of oxidised DNA lesions may explain the synthetic lethal relationship between MSH2 deficiency and POLB. To investigate whether the synthetic lethality associated with MLH1 deficiency and POLG inhibition could also due to an accumulation of oxidative damage, we determined the levels of 8-OHdG accumulation in MLH1 deficient cells depleted of POLG. This analysis showed that a significant increase in this DNA lesion also occurred in MLH1 deficient HCT116 cells upon POLG silencing (FIG. 9D). Consistent with our hypothesis, no significant increase was observed in the similarly transfected MLH1 proficient cell line, HCT116+Chr3.

POLB has been identified as one of the main nuclear DNA polymerases. In contrast, the human POLG gene encodes the catalytic subunit of what is believed to be the only DNA polymerase active in mitochondria. In light of this, we hypothesised that the difference in MSH2/POLB and MLH1/POLG synthetic lethalities may be explained by 8-OHdG accumulation in either the nucleus or mitochondria. To address this, we transfected MSH2 or MLH1 proficient and deficient cells with control, POLB or POLG siRNA as detailed above. However, instead of isolating and analysing total DNA, we fractionated cells into mitochondrial and nuclear fractions and extracted DNA. 8-OHdG accumulation was then quantified in these samples using an ELISA (FIGS. 9E & F). Increased 8-OHdG accumulation was observed in the mitochondrial DNA fraction from the MLH1 deficient cells transfected with POLG siRNA, whereas no significant accumulation of this lesion was observed in the nuclear DNA fraction. POLE silencing in MLH1 deficient cells did not increase nuclear or mitochondrial 8-OHdG levels. In contrast, mitochondrial and nuclear DNA extracts isolated from MSH2 deficient cells transfected with POLG siRNA showed no increase in 8-OHdG accumulation. However, nuclear DNA from MSH2 deficient cells transfected with POLE siRNA demonstrated a significant increase in 8-OHdG while no increase was observed in the DNA isolated from mitochondria. The efficiency of nuclear-mitochondrial fractionation was confirmed by western blot analysis (FIG. 9G). We conclude that MSH2 and POLB are individually redundant for 8-OHdG repair in the nucleus, whilst together they are non-redundant for this repair. Likewise MLH1 and POLG are individually redundant for 8-OHdG repair in mitochondria but together they are non-redundant.

We focussed in more detail on the MSH2-POLB synthetic lethal phenotype. We addressed the mechanism underlying the increased POLB expression in the context of MSH2 deficiency. In the absence of MSH2, it seems possible that oxidative damage itself rises and it is this that causes a compensatory increase in POLB levels. Therefore, to investigate the possibility that a rise in oxidative damage might be responsible for the observed increase in POLB transcript levels, MSH2 deficient and proficient cells were treated with the oxidising agent H₂0₂ and POLE mRNA levels measured shortly thereafter (FIG. 9H). In both MSH2 deficient and proficient cells, POLE mRNA levels were induced by H₂0₂ treatment consistent with our hypothesis. It is of note that the basal level of POLB mRNA expression in MSH2 deficient cells was equivalent to that in MSH2 proficient cells treated with H₂0₂ (FIG. 9H). This latter observation reinforces our previous data showing an elevated level of persistent oxidative DNA damage in MSH2 deficient cells, as shown by 8-OHdG ELISA and immunofluorescence detection.

### OGG1 Cleavage Activity and Expression is Decreased in the Absence of POLB Expression

Our data suggested that MSH2-POLB synthetic lethality correlates with an increase in oxidised DNA, as measured by both ELISA and immunofluorescent detection of 8-OHdG residues. The role of POLB in the repair of these lesions is well established; POLE acts downstream of the glycosylase OGG1, which removes the oxidised base, after which POLB contributes to the removal of the remaining sugar backbone and the polymerisation of new DNA in its place. Given that POLB acts downstream of the removal of the oxidised base detected by our ELISA and immunofluorescent methods, it was unclear why high levels of this lesion were detected in the context of MSH2 deficient cells transfected with POLB siRNA.

However, given that multiple components of molecular pathways are frequently coregulated to enable their coordination in complex molecular processes, we reasoned that reducing POLB levels might affect the activity of OGG1, therefore modulating the repair of the 8-OHdG lesion. Recent work has shown that other components of the BER pathway are co-regulated; the scaffold protein XRCC1 is required for recruitment of POLB and formation of the BER repair complex and XRCC1 deficiency leads to destabilization of BER proteins. Therefore, to investigate the possibility that POLE inhibition resulted in reduced OGG1 activity we used an in vitro OGG1 activity assay. HeLa cells were transfected with either a control siRNA, OGG1 siRNA or siRNA targeting POLB. Protein extracts were prepared from all transfected cells and incubated with a 23 base oligonucleotide containing 8-OHdG at its 11th base, labeled with 32P at its 5′ end, and annealed to its complementary strand (containing dC at the opposite base position to the 8-OHdG). The oligonucleotide strands were electrophoresed on a denaturing gel and the cleaved product was detected by autoradiography (FIG. 10A). As expected, extracts from HeLa cells transfected with control siRNA caused cleavage of the 8-OHdG radiolabelled oligonucleotide, resulting in a labeled 10 base cleavage product (FIG. 10B), suggestive of normal OGG1 activity. However, HeLa cells similarly transfected with POLB siRNA exhibited a significant decrease in OGG1 mediated cleavage of 8-OHdG, similar to that observed upon silencing of OGG1 itself. These data suggested that the OGG1 mediated cleavage of 8-OHdG is dependent on POLB expression.

To examine the level at which POLB expression levels control OGG1 activity, we transfected cells with either control, POLB or OGG1 siRNA and measured OGG1 protein levels by western blotting (FIG. 10C). OGG1 protein levels were significantly reduced in cells transfected with POLE siRNA but not in cells transfected with a control siRNA. Taken together, this analysis suggested that in the absence of POLE expression, the OGG1 dependent cleavage of 8-OHdG is abrogated via a decrease in the expression of OGG1.

### POLB Silencing Leads to Decreased OGG1 Expression Via CHIP-Mediated Degradation

Our data suggested that the expression of the OGG1 glycosylase is dependent on POLB expression. Previously it has been shown that OGG1 interacts with the BER protein XRCC1, and this interaction results in a 2-3 fold stimulation of OGG1 activity. By analogy, we investigated whether POLE and OGG1 physically interacted. We performed immunoprecipitation of POLE from HeLa cell extracts, followed by immuno-detection of OGG1. Immunoblot analysis demonstrated an interaction between POLB and OGG1 (FIG. 11A). Recent work has shown that BER proteins such as POLB, Ligase III and XRCC1, when not involved in repair complexes are ubiquitylated by the carboxyl terminus of Hsc70 interacting protein (CHIP) and due to this are degraded by the proteasome. It seemed possible, therefore, that upon destabilization of the OGG1/POLB repair complex by POLB inhibition, OGG1 was degraded in a CHIP dependent manner. To assess this, HeLa cells were transfected with either control, POLB or POLE and CHIP siRNA together (FIG. 11B). Protein lysates from the transfected cells were immunoblotted for OGG1 expression. As before, upon POLB silencing, OGG1 expression was decreased. However, the combined silencing of both POLB and CHIP resulted in restoration of OGG1 expression, which suggested that downregulation of OGG1 by POLB is dependent upon CHIP expression. It has been shown that POLB, XRCC1 and Ligase III protein levels are increased following transfection with CHIP siRNA, suggesting that these proteins become degraded less efficiently. Here we also observed increased levels of POLB expression upon CHIP silencing and similarly OGG1 expression is increased in the absence of CHIP, further supporting the role of CHIP in the degradation of OGG1. Finally, treatment of POLB silenced cells with the proteasomal inhibitor MG132 restored the expression of OGG1 (FIG. 11C).

Therefore, our data suggest that upon POLB silencing, OGG1 expression is reduced due to ubiquitination by CHIP. Taken together, our results indicate that POLB and OGG1 form a complex to repair oxidative damage such as 8-OHdG lesions. However, upon inhibition of POLB expression, this interaction is abrogated, enabling the degradation of OGG1 by CHIP.

### Discussion

Multiple DNA repair pathways collaborate to repair the spectrum of DNA lesions caused by endogenous and exogenous DNA damage and these interactions may be exploited therapeutically (Lord et al., 2006). Exploitation of this phenomenon has recently been demonstrated by the interplay of SSB repair by the enzyme PARP1 and homologous recombination by BRCA1 and BRCA2. In this study, an additional DNA repair pathway interaction is described resulting from loss of the primary BER polymerase β in a MMR-deficient background, illustrated in the schematic model in FIG. 6. Previous studies using animal models deficient in BER activity accumulate more damage in response to oxidative stress, establishing a role for BER and consequently Polβ in the repair of oxidative damage (Cabelof et al., 2003). In support of this hypothesis, it has been demonstrated in fibroblasts that lipopolysaccharide-induced oxidative damage induces Polβ levels and that more damage accumulates in the Polβ null fibroblasts (Chen et al., 1998). However, a variety of repair mechanisms could exist to remove deleterious oxidative damage. Here evidence is provided that in the absence of functional MSH2, POLβ expression is upregulated and that this is necessary for repair of 8-OHdG oxidative lesions. It has been reported that some human cancer cell lines and cancer tissues such as lung (Erhola et al., 1997), renal (Okamoto et al., 1994), and colorectal carcinoma (Kondo et al., 1997 and Kondo et al., 2000) show higher levels of DNA oxidation compared to their non-tumourous counterparts, as assessed not only by an increase in 8-OHdG levels but also by increases in 8-OHdG lyase activity which is specifically observed in human colorectal carcinoma (Kondo et al., 2000). Consequently, the use of POLβ inhibitors for treatment of MSH2-deficient cancers has increased therapeutic potential. A number of compounds which are considered specific POLf3 inhibitors have been identified, including koetjapic acid (Sun et al., 1999), pamoic acid (Horton et al., 2004), prunasin (Mizushina et al., 1999), solanapyrone A (Mizushina et al., 2002), trans-communic acid, mahureone A, and also masticadienonic acid (Boudsocq et al., 2005) which with an 1050 of 8″ is the most potent POLβ inhibitor to date. Whilst the Polβ(−/−) mouse is embryonic lethal (Gu et al., 1994), mouse fibroblasts with a deletion of the Polβ gene exhibit moderate hypersensitivity to monofunctional alkylating agents, such as methyl methanesulfonate (Horton and Wilson, 2006). Thus, further investigation into available POLβ inhibitors along with the development of new inhibitors with decreased toxicity and increased POLb specificity, shows growing therapeutic promise.

Overexpression of DNA polymerases has been widely reported in cancer. In particular, POLβ was shown to be frequently overexpressed in uterus, ovary, prostate and stomach tumours. (Albertella et al., 2005). Colon and breast adenocarcinomas also exhibited increased levels of POLβ expression. (Srivastava et al., 1999). In this study we demonstrate that upon loss of MSH2, expression of POLβ is increased. This suggests that lack of MMR activity, activates POLβ as a compensatory mechanism, suggesting interplay between these distinct DNA repair pathways. Previous studies also show compensatory interactions between DNA repair pathways, most notably the upregulation of non-conservative DSB repair pathways in the absence of conservative DSB repair in BRCA2 deficient cells.

A cell-based compound screen targeting the MMR pathway was carried out in cells lacking a functional MSH2 protein. This revealed complementary information with those obtained using an siRNA approach to disable POLβ function. Down regulation of POLβwas associated with increased 8-OHdG accumulation. This potentiation of damage was very similar to the effects of methotrexate, menadione and parthenolide seen in our study. While the current findings provide important identification of POLβ as an anticancer drug target, our data also suggests that such compound screens can be highly informative and likely compliment target-based siRNA screening. Clearly, these findings have significant translational implications.

We show here that deficiencies in the major MMR proteins, MSH2 and MLH1 are synthetically lethal with silencing of distinct DNA polymerases. Loss of expression of these MMR proteins is associated with elevated expression of the respective polymerases. We hypothesise that increased POLE or POLG expression compensates for the reduction in 8-OHdG repair caused by MSH2 or MLH1 deficiency. Our data support the hypothesis that a defective MMR pathway results in an upregulation of the POLB-dependent, OGG1 mediated repair, of 8-OHdG lesions. However, polymerase inhibition superimposed on MMR deficiency, increases the level of 8-OHdG lesions, which might then pass a threshold that is inconsistent with viability (FIG. 12).

These synthetic lethal relationships suggest novel therapeutic approaches. The use of POLB inhibitors for treatment of MSH2-deficient cancers has considerable clinical potential. A number of somewhat specific but not very potent POLB inhibitors have been identified, including koetjapic acid (Sun et al., 1999), pamoic acid (Hu et al., 2004), prunasin (Mizushina et al., 1999), solanapyrone A (Mizushina et al., 2002), trans-communic acid, mahureone A, and also masticadienonic acid (Boudsocq et al., 2005) which with an 1050 of 8 μM, is the most potent POLB inhibitor identified to date. While the PolB null mouse is embryonically lethal (Gu et al., 1994), mouse fibroblasts with a deletion of the PolB gene are viable but exhibit moderate hypersensitivity to monofunctional alkylating agents, such as methyl methanesulfonate (Horton and Wilson, 2007). Therefore, the development of new inhibitors with decreased toxicity and increased POLB potency may have considerable therapeutic promise.

It has been reported that the level of DNA polymerases is significantly elevated in some human adenocarcinomas and tumor cell lines relative to the expression level in normal tissues and cells (Albertella et al., 2005; Srivastava et al., 1999). Here, we show the upregulation of POLB in MSH2 deficient tumour cells and POLG in MLH1 deficient tumor cells. We also observe a significant increase in POLB and POLG expression in MMR deficient colorectal tumours. This supports the notion that in both cancer cell lines and in patient tumours, loss of MMR function is associated with a compensatory upregulation in polymerase expression. Upregulation of POLB and POLG in the absence of MMR function may therefore provide novel biomarkers for MMR deficiency. In summary, our data supports the hypothesis that a defective MMR pathway is a determinant of decreased cellular viability in the absence of BER polymerases involved in oxidative damage repair. We suggest that the mechanistic basis for this synthetic lethal interaction is the accumulation of oxidized DNA lesions. It is, therefore, possible that the use of oxidative damage-inducing drugs may also be a potential therapeutic approach for the treatment of MMR deficient cancers.

In general, the standard adjuvant treatment of colorectal cancers involves 5-fluorouracil (5-FU)-based chemotherapy regimens (Chau & Cunningham, 2006). Leucovorin is often used in combination and enhances the effect of 5-FU on inhibiting thymidylate synthase. Studies suggest that, in addition to tumour stage, the presence of microsatellite instability (MST) due to MMR deficiency within a patient's colorectal tumour may predict survival after 5-FU treatment (Jover et al., 2006). These studies indicate that patients whose tumours are MMR defective derived no survival benefit from 5-FU treatment, whereas patients whose tumours had intact MMR had improved survival. In vitro studies with 5-FU indicate that MMR-defective colon cancer cells are increasingly resistant when compared with MMR-proficient cells (Arnold et al., 2003). In addition to the effects of 5-FU on thymidylate synthetase, some 5-FU is incorporated into DNA, indicating recognition by the MMR system suggesting that the MMR pathway can selectively recognize 5-FU and mediate chemosensitivity in cell lines and tumours (Jo and Carethers, 2006). Therefore, identification of a selective therapeutic target for MSH2-deficient cancers such as methotrexate, may benefit treatment impaired by MMR associated drug resistance.

The promise for use of methotrexate in the treatment of MSH2-deficient cancers is further supported by the fact that this agent is currently in use in the clinic. Studies have determined that adjuvant sequential modulation of 5-FU by methotrexate results in similar outcome as the standard modulation of 5-FU by leucovorin, in patients with unknown MMR status (Sobrero et al., mos). Therefore suggesting that in a trial with patients selected for, with respect to MSH2 status, the potential of methotrexate as a third-line therapeutic agent, where the standard treatment has shown no benefit, is significantly promising. Furthermore, HNPCC patients exhibit distinct clinical definition, with tumours predominantly right-sided, often mucinous, poorly differentiated, and may be distinguished by peritumoural lymphocytic reaction. HNPCC adenomas tend to be villous and have a component of high-grade dysplasia (Half and Bresalier, 2004). Thus offering a good clinical marker for patients who may benefit from methotrexate treatment.

In summary, the data provided herein supports the hypothesis that a defective MMR pathway is a determinant of decreased cellular viability in the absence of oxidative damage associated POLβ. The utility of parallel compound and RNAi screens was validated by the discovery of a compound, methotrexate that targets POLβ expression, a novel MSH2 synthetic lethal partner. Resistance of MMR deficient tumours to standard colon cancer drug treatments such as 5-FU, further argues for the use of methotrexate as a specific therapeutic agent in the treatment of MSH2-deficient cancers.

### REFERENCES

All publications, patent and patent applications cited herein or filed with this application, including references filed as part of an Information Disclosure Statement are incorporated by reference in their entirety.
Aaltonen et al, 1993, Science, 260; 812-6.
Aaltonen et al., 1994, Cancer Res., 54, 1645-8.
Akiyama et al., Germ-line mutation of the hMSH6/GTBP gene in an atypical hereditary nonpolyposis colorectal cancer kindred. Cancer Res., (1997) 57, 3920-3.
Albertella et al., (2005) The overexpression of specialized DNA polymerases in cancer. DNA Repair. 4, 583-593.
Argueso et al., (2002) Analysis of conditional mutations in the Saccharomyces cerevisiae MLH1 gene in mismatch repair and in meiotic crossing over. Genetics 160, 909-921.
Arnold et al., (2003) Role of hMLH1 promoter hypermethylation in drug resistance to 5-fluorouracil in colorectal cancer cell lines. Int. J. Cancer 106, 66-73.
Bergoglio et al., (2002) Deregulated DNA polymerase β induces chromosome instability and tumourigenesis. Cancer Res. 62, 3511-3514.
Bettstetter et al., (2007) Distinction of Hereditary Nonpolyposis Colorectal Cancer and Sporadic Microsatellite-Unstable Colorectal Cancer through Quantification of MLH1 Methylation by Real-time PCR. Clin. Cancer Res. 13, 3221-3228.
Boudsocq et al., (2005) Modulation of Cellular Response to Cisplatin by a Novel Inhibitor of DNA Polymerase β. Mol. Pharmacol. 67, 1485-1492.
Cabelof et al., (2003) Base Excision Repair Deficiency Caused by Polymerase β Haploinsufficiency Accelerated DNA Damage and Increased Mutational Response to Carcinogens. Cancer Res., 63, 5799-5807.
Chau & Cunningham, (2006) Adjuvant therapy in colon cancer—what, when and how?, Annals of Oncology 17, 1347-1359.
Chen et al., (1998) Up-regulation of base excision repair correlates with enhanced protection against a DNA damaging agent in mouse cell lines. Nucl. Acids Res. 26, 2001-2007.
Colussi et al., (2002) The mammalian mismatch repair pathway removes DNA 8-oxodGMP incorporated from the oxidized dNTP pool. Curr. Biol., 12, 912-918.
Cojocel et al., (2006) Mutagenic and carcinogenic potential of menadione. Neoplasma 53, 316-323.
Cunningham et al., (1998). Hypermethylation of the hMLH1 promoter in colon cancer with microsatellite instablitity. Cancer Res. 58, 3455-3460.
DeWeese et al., (1998) Mouse embryonic stem cells carrying one or two defective Msh2 alleles respond abnormally to oxidative stress inflicted by low-level radiation. Proc. Natl. Acad. Sci. U.S.A. 95, 11915-11920.
Dickins et al, (2005). Probing tumor phenotypes using stable and regulated synthetic microRNA precursors. Nat. Genet. 37, 1289-1295.
Erhola et al., (1997) Biomarker evidence of DNA oxidation in lung cancer patients: association of urinary 8-hydroxy-2′-deoxyguanosine excretion with radiotherapy, chemotherapy, and response to treatment. FEBS Lett., 409, 287-291.
Felton et al., (2007) Constitutive deficiency in DNA mismatch repair. Clinical Genetics 71, 483-498.
Goodsell, (1999) The Molecular Perspective: Methotrexate. Stem Cells 17, 314-315.
Gu et al., (1994) Deletion of a DNA polymerase beta gene segment in T cells using cell type-specific gene targeting. Science 265, 103-106.
Half & Bresalier, (2004) Clinical Management of Hereditary Colorectal Cancer Syndromes. Clinical definition. Curr. Opin. Gastroenterol., 20, 32-42.
Horton & Wilson, (2006) Hypersensitivity phenotypes associated with genetic and synthetic inhibitor-induced base excision repair deficiency. DNA Repair 6, 530-543.
Hsieh, (2001) Molecular mechanisms of DNA mismatch repair. Mutation Research 486, 71-87.
Hu et al., (2004) Identification of Small Molecule Synthetic Inhibitors of DNA Polymerase by NMR Chemical Shift Mapping. J Biol. Chem., 279, 39736-39744.
Ionov et al, 1993, Nature, 363, 558-61.
Jacob & Praz, (2002) DNA mismatch repair defects: role in colorectal carcinogenesis. Biochimie, 84, 27-47.
Jascur & Boland, (2006) Structure and function of the components of the human DNA mismatch repair system. Int. J. Cancer. 119, 2030-2035.
Jiricny, (2006) The multifaceted mismatch-repair system. Nat. Rev. Mol. Cell Biol. 7, 335-346.
Jo & Carethers, (2006) Chemotherapeutic implications in microsatellite unstable colorectal cancer. Cancer Biomarkers 2, 51-60.
Jover et al., (2006) Mismatch repair status in the prediction of benefit from adjuvant fluorouracil chemotherapy in colorectal cancer. Gut 55, 848-855.
Kaelin, (2005) The concept of synthetic lethality in the context of anticancer therapy. Nat. Rev. Cancer 5, 689-698.
Khare & Eckert, (2002) The proofreading 3′-5′ exonuclease activity of DNA polymerases: a kinetic barrier to translesion DNA synthesis. Mutation Research 510, 45-54.
Kondo et al., (1999) Persistent oxidative stress in human colorectal carcinoma, but not in adenoma. Free Radic. Biol. Med. 27, 401-410.
Kondo et al., (2000) Overexpression of the hOGG1 Gene and High 8-Hydroxy-2′-deoxyguanosine (8-OHdG) Lyase Activity in Human Colorectal Carcinoma Regulation Mechanism of the 8-OHdG Level in DNA. Clin. Cancer Res. 6, 1394-1400.
Kunkel & Bebenek, (2000) DNA Replication Fidelity. Ann. Rev. Biochem. 69, 497-529.
Kurdi et al., (2007) Parthenolide induces a distinct pattern of oxidative stress in cardiac myocytes. Free Radic. Biol. Med. 42, 474-481.
Lehman, (2006) Translesion synthesis in mammalian cells. Exp. Cell Res., 312, 2673-2676.
Liu et al., 1996, Nat. Med., 2, 169-74.
Mackay, et al, (2000). Reduced MLH1 expression in breast tumors after primary chemotherapy predicts disease-free survival. J Clin Oncol 18, 87-93.
Miyaki et al., (1997) Germline mutation of MSH6 as the cause of hereditary nonpolyposis colorectal cancer. Nat. Genet., 17, 271-2.
Mitchell et al., (2002) Mismatch Repair Genes hMLH1 and hMSH2 and Colorectal Cancer: A HuGE Review. Am. J. EPID 156, 885-902.
Mizushina et al., (1999) The cyanogenic glucoside, prunasin (D-mandelonitrile-beta-D-glucoside), is a novel inhibitor of DNA polymerase beta. J. Biochem. 126, 430-436.
Mizushina et al., (2002) A plant phytotoxin, solanapyrone A, is an inhibitor of DNA polymerase beta and lambda. J. Biol. Chem. 277, 630-638.
Morrison et al., (1993) Pathway correcting DNA replication errors in Saccharomyces cerevisiae. EMBO J., 12, 1467-1473.
Navi et al., (2006) Muir-Torre syndrome. Dermatol. Online J. 12, 4.
Okamoto et al., (1994) Formation of 8-hydroxy-2′-deoxyguanosine and 4-hydroxy-2-nonenal-modified proteins in human renal-cell carcinoma. Int. J. Cancer, 58, 825-829.
Paddison et al. (2004). Cloning of short hairpin RNAs for gene knockdown in mammalian cells. Nat Methods 1, 163-167.
Pavlov et al., (2001) Mutator effects of overproducing DNA polymerase eta (Rad30) and its catalytically inactive variant in yeast. Mutat. Res., 478, 129-139.
Pavlov et al., (2006) Roles of DNA polymerases in replication, repair, and recombination in eukaryotes. Int. Rev. Cytol., 255, 41-132.
Peltomaki et al., (1993). Mutations of a mutS homolog in hereditary nonpolyposis colorectal cancer. Cell 75, 1215-1225.
Poley et al., (2007) Biallelic germline mutations of mismatch-repair genes. Cancer, 109, 2349-2356.
Potocnik et al., (2001). Causes of microsatellite instability in colorectal tumors: implications for hereditary non-polyposis colorectal cancer screening. Cancer Genet. Cytogenet. 126, 85-96.
Rajamani et al., (2006) Oxidative stress induced by methotrexate alone and in the presence of methanol in discrete regions of the rodent brain, retina and optic nerve. Toxicol. Lett., 165, 265-273.
Scott et al., (2007) Familial T-cell non-Hodgkin lymphoma caused by biallelic MSH2 mutations. J. Med. Genet., 44, e83.
Sobol et al., (1996) Requirement of mammalian DNA polymerase-beta in base-excision repair. Nature 379, 183-186.
Sobrero et al., (2005) Adjuvant sequential methotrexate→5-fluorouracil vs 5-fluorouracil plus leucovorin in radically reseacted stage III and high-risk stage II colon cancer. Brit. J. Cancer, 92, 24-29.
Srivastava et al., (1999) DNA polymerase β expression differences in selected human tumours and cell lines. Carcinogenesis 20, 1049-1054.
Sun et al., (1999) DNA Polymerase β Inhibitors from Sandoricum koetjape. J. Nat. Prod. 62, 1110-1113.
Thompson, (2004) DNA Oxidation Products, Antioxidant Status, and Cancer Prevention. J. Nutr. 134, 3186S-3187S.
Tran et al., (1999) The 3′-5′, exonucleases of DNA polymerases and the 5′-3′ exonuclease Exol have major roles in postreplication mutation avoidance in Saccharomyces cerevisiae. Mol. Cell Biol. 19, 2000-2007.
Tran et al., (1997) Hypermutability of homonucleotide runs in mismatch repair and DNA polymerase proofreading yeast mutants. Mol. Cell Biol., 17, 2859-2865.
Umar et al., (1997) Correction of hypermutability, N-methyl-N′-nitro-N-nitrosoguanidine resistance, and defective DNA mismatch repair by introducing chromosome 2 into human tumour cells with mutations in MSH2 and MSH6. Cancer Res., 57, 3949-3955.
Yano et al., (2007) Prognosis in patients with hepatocellular carcinoma correlates to mutations of p53 and/or hMSH2 genes. Eur. J. Cancer 43, 1092-1100.
Yoshimura et al., (2006) Vertebrate POLQ and POL β Cooperate in Base Excision Repair of Oxidative DNA Damage. Mol. Cell 24, 115-125.
Zhao et al., (2005) Transcriptional upregulation of DNA polymerase beta by TEIF. Biochem. Biophys. Res. Commun. 333, 908-916.
Brinkman et al (1998). Adverse effects of reverse transcriptase inhibitors: mitochondrial toxicity as common pathway. AIDS 12, 1735-1744
Cherrington et al, (1994). Kinetic analysis of the interaction between the diphosphate of (S)-1-(3-hydroxy-2-phosphonylmethoxypropyl)cytosine, ddCTP, AZTTP, and FIAUTP with human DNA polymerases beta and gamma. Biochem Pharmacol 48, 1986-1988.
Lewis et al, (1994). Cardiac mitochondrial DNA polymerase-gamma is inhibited competitively and noncompetitively by phosphorylated zidovudine. Circ Res 74, 344-348.
Mazzucco et al (2008). Entecavir for treatment of hepatitis B virus displays no in vitro mitochondrial toxicity or DNA polymerase gamma inhibition. Antimicrob Agents Chemother 52, 598-605.
Sasaki et al (2008). DNA polymerase gamma inhibition by vitamin K3 induces mitochondria-mediated cytotoxicity in human cancer cells. Cancer Sci 99, 1040-1048.

1.-27. (canceled)

28. A method of screening for agents useful in the treatment of a DNA mismatch repair (MMR) pathway deficient cancer, the method employing first and second cell lines, wherein the first cell line is deficient in a component of the DNA mismatch repair (MMR) pathway and the second cell line is proficient for said component of the DNA mismatch repair (MMR) pathway, the method comprising:
(a) contacting the first and second mammalian cell lines with at least one candidate agent;
(b) determining the amount of cell death in the first and second cell lines; and
(c) selecting a candidate agent which is synthetically lethal in the first cell line.

29. The method of claim 25, wherein the cell lines are cancer-derived cell lines.

30. The method of claim 25, wherein the cell lines are a MMR-deficient murine stem cell line.

31. The method of claim 28, wherein the first and second cells lines are isogenically matched.

32. The method of claim 28, wherein the MMR-deficient cell line is produced by RNA interference of a gene in the MMR pathway.

33. The method of claim 28, wherein step (c) comprises selecting candidate agents that do not cause a substantial amount of cell death in the second cell line.

34. The method of claim 28, wherein the cells are Hec59 cells.

35. The method of claim 28, further comprising the step of determining whether a candidate agent selected in step (c) is an inhibitor of a protein target selected from DNA polymerase POLβ, DNA polymerase POLy, telomerase transcriptional element integrating factor (TEIF or SCYL1) and/or dihydrofolate reductase (DHFR).

36. The method of claim 28, further comprising the step of contacting a candidate agent selected in step (c) with a cell line deficient in the component of the DNA mismatch repair (MMR) pathway to determine whether the candidate agent causes 8-OHdG to accumulate in the cell line.

37.-53. (canceled)

54. A method of treating an individual having a DNA mismatch repair (MMR) deficient cancer, the method comprising administering a therapeutically effective amount of an inhibitor of DNA polymerase POLβ to the individual.

55. A method of treating an individual having a DNA mismatch repair (MMR) deficient cancer, the method comprising administering a therapeutically effective amount of an inhibitor of DNA polymerase POLβ, DNA polymerase POLy, telomerase transcriptional element integrating factor (TEIF or SCYL1) and/or dihydrofolate reductase (DHFR) to the individual.

56.-60. (canceled)


SEQUENCE LISTING (22 sequences)

SEQ ID NO: 1 - DNA, 97 bp, Artificial Sequence
  Synthetic Sequence
tgctgttgacagtgagcgcctcagtgaattaagagaaatatagtgaagccacagatgtatatttctcttaattcactgagatgcctactgcctcgga

SEQ ID NO: 2 - DNA, 21 bp, Artificial Sequence
  siRNA target sequence
cacgagcaaatcttcgggcaa

SEQ ID NO: 3 - DNA, 21 bp, Artificial Sequence
  siRNA target sequence
cagatgcgggtcacacctaaa

SEQ ID NO: 4 - DNA, 21 bp, Artificial Sequence
  siRNA target sequence
caagatattgtactaaatgaa

SEQ ID NO: 5 - DNA, 21 bp, Artificial Sequence
  siRNA target sequence
tacgagttcatccatcaattt

SEQ ID NO: 6 - DNA, 21 bp, Artificial Sequence
  siRNA target sequence
accgggaacatcaggctttaa

SEQ ID NO: 7 - DNA, 21 bp, Artificial Sequence
  siRNA target sequence
gcggtttattaagctcttcta

SEQ ID NO: 8 - DNA, 21 bp, Artificial Sequence
  siRNA target sequence
atccatttaggtgctgagtta

SEQ ID NO: 9 - DNA, 21 bp, Artificial Sequence
  siRNA target sequence
ctggttgtgagcattcgtgta

SEQ ID NO: 10 - DNA, 21 bp, Artificial Sequence
  siRNA target sequence
ccgcatcatcctctgtacaaa

SEQ ID NO: 11 - DNA, 21 bp, Artificial Sequence
  siRNA target sequence
ccgcctctccattgacctgaa

SEQ ID NO: 12 - DNA, 21 bp, Artificial Sequence
  siRNA target sequence
cgggacctacacctcaggaaa

SEQ ID NO: 13 - DNA, 21 bp, Artificial Sequence
  siRNA target sequence
caccgtgtgggcgaggcctta

SEQ ID NO: 14 - DNA, 20 bp, Artificial Sequence
  siRNA target sequence
ccgcggagcguagagaggga

SEQ ID NO: 15 - DNA, 19 bp, Artificial Sequence
  siRNA target sequence
gcauugaggccaagcacga

SEQ ID NO: 16 - DNA, 21 bp, Artificial Sequence
  siRNA target sequence
tacggagaaactgaactgaga

SEQ ID NO: 17 - DNA, 21 bp, Artificial Sequence
  siRNA target sequence
aacctccacaaggagctcatt

SEQ ID NO: 18 - DNA, 21 bp, Artificial Sequence
  siRNA target sequence
cccgttgggaatatacctcaa

SEQ ID NO: 19 - DNA, 21 bp, Artificial Sequence
  siRNA target sequence
caaccgctttgtagaaaccaa

SEQ ID NO: 20 - DNA, 21 bp, Artificial Sequence
  siRNA target sequence
cccatgggctatcaacttaat

SEQ ID NO: 21 - DNA, 21 bp, Artificial Sequence
  siRNA target sequence
tccaggcatgcttgtgttgaa

SEQ ID NO: 22 - DNA, 19 bp, Artificial Sequence
  siRNA target sequence
catgcctgatccgctagtc